(19) United States
(12) Patent Application Publication
(10) Pub. No.: US 2004/0142379 A1
(43) Pub. Date: Jul. 22, 2004

(54) Affinity fishing for ligands and proteins receptors

(75) Inventors: Phaedria Marie St. Hilaire, Copenhagen (DK); Haifeng Yin, Frederiksberg (DK); Sheryl Surve, Lund (SE)

Correspondence Address: BROWDY AND NEIMARK, P.L.L.C., 624 NINTH STREET, NW, SUITE 300, WASHINGTON, DC, 20001-5303, US

(73) Assignee: Carlsberg Research Laboratory, Copenhagen (DK)

(21) Appl. No.: 10/346,737

(22) Filed: Jan. 16, 2003

(51) Int. Cl. G01N 33/53; G01N 33/543

(52) U.S. Cl. 435/7.1; 436/518

(57) ABSTRACT

The invention provides putative “drugable” protein targets and actively binding ligands identified in an efficient and reproducible process by determining the affinity of protein mixtures to libraries of ligand compounds of defined size and composition. The libraries are used to isolate and identify previously unknown corresponding protein-ligand binding pairs from a mixture of proteins and a library of compounds, and are particularly useful to identify differentially selective protein-ligand binding pairs, for example, representing a single physiological state or several varied but related states, such as disease versus normal conditions.

### FIELD OF THE INVENTION

The present invention relates to the use of proteomics and combinatorial chemistry in combination to provide powerful tools and methods for the drug discovery process.

### BACKGROUND OF THE INVENTION

The recognition and binding of ligands to receptors is a fundamental process providing the molecular architecture of most biological phenomena, including immune recognition, cell signaling, catalysis, metastasis, and pathogenic invasion of a host's cells. Consequently, there has been a driving impetus, both in basic and applied research, to identify and characterize receptors and corresponding ligands with the intent to elucidate biological pathways and to develop therapeutics for amelioration of various disease states.

This selective interaction between a particular protein and a ligand is the cornerstone of the drug discovery process. Traditionally, the search for such receptor/ligand pairs has been carried out in a sequential manner such that the identity of a protein is first determined from a genomic/proteomic standpoint (gene knockout, gene sequence analysis, proteomics). Once a protein of interest has been identified and validated as a drug target, suitable ligands can be identified using rational drug design, natural product screening, or screening of putative ligand libraries. Alternatively, a particular protein can be purified from a mixture after a particular ligand known to bind to that family of proteins is identified. See, for example, U.S. Pat. Nos. 5,834,318, and 5,783,663, and published PCT patent application 9838329A1.

In the context of receptor-ligand interactions in the pharmaceutical industry, such sequential approaches are not ideal. Designing ligands for drug targets derived solely from analysis and comparison of an organism's genome or proteome can fail to achieve a desired drug effect because the selected target is not “drugable.” The target may prove unsuitable for use as a therapeutic drug due to lack of specificity, toxicity, and the like. Traditional approaches for drug screening have proven relatively effective, but are time consuming and inefficient. In addition, little consideration is given to the potential toxicity of the drug during the initial phases of traditional selection. These inefficiencies lead to failures in later clinical trial, as well as unnecessary development time and expense. Therefore, approaches to matching receptor-ligand interactions at an early stage in the drug discovery program are highly advantageous. The invention described herein achieves this purpose by rapidly matching unknown proteins with unknown ligands, thus short-listing the number of potential drug targets and their putative drug leads in a single process. This invention, furthermore, provides information on the specificity, cross-reactivity, potential toxicity and other characteristics of the drug lead as well as data relating to possible combination therapies. For example, from the ligand-protein matches it is immediately clear whether a ligand interacts with more than one protein, and whether one of the proteins is of vital importance for the functioning of the cell (potential toxicity effect). It will also be apparent whether several ligands interact with a singular protein, thus increasing the number of potential drug leads and possibilities for combination therapy.

An alternative approach to identifying ligands and receptors when the precise nature of the ligand and target are unknown has been described by some researchers using phage display. Phage displaying surface peptides target specific receptors in particular organs when applied in an in vivo system (see, for example, U.S. Pat. Nos. 5,622,699 and 6,306,365). The phage are then recovered from the organ, the peptide identified, and the receptor subsequently isolated and identified using affinity chromatography. This approach is largely limited to libraries of peptide ligands consisting of the 20 genetically-encoded amino acids, and cannot take advantage of useful synthetic amino acids. In addition, since the targeting takes place in vivo, proteolysis of some peptide ligands by adventitious proteases will take place, thus reducing the number of putative ligands, and hence the number of targets that can be identified. Furthermore, it is also essential for phage to be endocytosed by the cell in order to target cytosolic proteins. The primary use of the phage display process is to identify peptides that can be used to deliver drugs to specific cells, organs, and tissues.

In an alternative to displaying peptide libraries on phage, peptide libraries can also be generated in mammalian cells using retroviral vectors (see for example, published PCT patent application WO 09638553 to Inoxell, U.S. Pat. No. 6,153,390 to Rigel, and related patents). Libraries of effector molecules (peptides, RNA molecules/ribozymes, or cDNA) are generated in cell lines that model a disease or cellular pathway. After the application of selective pressure and induction of the desired phenotype, the responsible effector molecule(s) is identified, and the corresponding cellular target(s) can be isolated using affinity chromatography and characterized. This approach is again restricted to naturally occurring oligomers. In addition, it is time consuming to develop the appropriate disease model cell. Furthermore, the peptide is expressed in a protein scaffold making it difficult to extrapolate to a small peptide/molecule drug.

The present invention capitalizes on progress made in the field of proteomics. For recent reviews discussing the state of this art see Peng et al., 2001, J. Mass Spectrom., 36: 1083-1091; and Yarmush et al., 2002, Annu. Rev. Biomed. Eng., 4: 349-373. In proteomics, the proteins of a cell are separated by 2-dimensional (2-D) gel electrophoresis and characterized by a combination of enzymatic digests and mass spectrometry (MS). When used to identify proteins that are potentially important in a diseased state, for example, 2-D gels displaying protein obtained from normal and abnormal states are compared and protein differences are identified. Proteins obtained from normal and abnormal samples can be differentially labeled (see for example, Unlu et al., 1997, Electrophoresis, 18: 2071-2077; and Gygi et al., 1999, Nat. Biotechnol., 17: 994-999). The differentially labeled proteins can be separated on a single 2-D gel before tryptic digestion and MS identification of the changed proteins as described, for example, in Unlu et al., 1997, Supra. Alternatively, the differentially labeled proteins can be first enzymatically digested and the peptides separated by liquid chromatography before MS analysis. See, for example, Gygi et al., 1999 Supra; and Washburn et al., 2001, Nat. Biotechnol., 19: 242-247. The use of two-dimensional gels for profiling an organism's proteome is not simple and is fraught with problems. The entire process from casting gels and protein solubilization to interpreting the protein patterns obtained poses numerous challenges. With careful attention to detail, individual laboratories might reproduce 2-D protein patterns; however, in practice, it is rare for different groups to obtain the same 2-D pattern, rendering comparison of data between laboratories difficult and the creation of shared databases largely pointless (see, Defrancesco, 1999, The Scientist, 13: 16). Furthermore, use of 2-D gels limits the size of proteins that can be separated. It is particularly difficult to isolate and identify membrane proteins and low abundance proteins by these techniques. The use of gel-free systems reduces the problems of size and abundance; however, in some cases, labeling procedures are limited as they require the presence of particular amino acids in a protein, and analysis of the mass spectra is difficult.

In a variation on proteomic profiling, proteins from a crude mixture can be captured on the surface of a chip, for example, a 2 mm chip, bearing any of a variety of affinity surfaces: antibodies, known protein receptors, nucleic acids, carbohydrates, and the like. Protein or proteins that bind to the chip can then be analyzed and identified, for example, using a technology called SELDI™ (surface-enhanced laser desorption/ionization) mass spectrometry. See for example, Davies et al., 1999, Biotechniques, 27: 1258-1261 and the world wide web (www) site: ciphergen.com. To utilize this technique, the immobilized binding partner must first be synthesized (peptide, carbohydrate, nucleic acid), isolated (protein receptor), or generated (antibody) before it is immobilized on the surface. Additionally, the immobilization procedure should not affect the nature and active conformation of the ligand. Thus, considerable effort can be expended to optimize immobilization for a particular set of ligands. This technique is also plagued by non-specific binding interactions, due in part to denaturing of the proteins in the crude mixture on the chip surface. Furthermore, relatively few binding partners can be immobilized on a single chip.

In an attempt to extend proteomic profiling to a wider array of compounds, a small, encoded soluble library (six compounds) was synthesized and screened with individual proteins in solution phase (see, for example, Winssinger et al., 2001, Angew. Chem. Int. Ed. Engl., 40: 3152-3155). The members of the library were laboriously encoded with a polynucleic acid tag (PNA tag) enabling the binding partners to be identified by hybridization to a DNA microarray. After identification of the ligand, the binding protein(s) can be purified by affinity purification using the identified ligand attached to a suitable support, and then identified using mass spectrometry. In this approach, the initial screening takes place in solution and is subject to many well-known problems. For example, as proteins bound to ligands are separated from proteins without ligands using gel-filtration chromatography, some ligands are lost, and only binding interactions that are extremely tight with very slow off rates (high-binding affinities) will be detected. Furthermore, some proteins may interact with the encoding PNA tag (wholly or partially) leading to a false positive or preventing hybridization and identification of a “true” positive binding pair. Finally, once an active ligand(s) is identified, the binding protein is identified using conventional affinity chromatography requiring resynthesis and immobilization of the ligand to a solid support, binding of the protein(s), and elution of the proteins(s) under the appropriate conditions, each procedure adding time and inefficiency to the selection process.

The present invention provides a novel, efficient, and effective process for identifying and matching ligands and putative drug targets with tremendous speed (lending itself to automation) and with few limitations as compared to known processes.

### SUMMARY OF THE INVENTION

In the process of the present invention, previously unknown, specific protein-ligand binding pairs are isolated and identified from a mixture of proteins and a ligand library by virtue of specific binding, isolation, and identification. In a preferred embodiment, a library of spatially separated ligands, immobilized on a solid support, is incubated with a mixture of proteins, such as proteins that have been isolated from cells, tissue, or organisms. The protein mixture can be labeled with a detection probe. After incubation of the immobilized ligands with the protein mixture, active ligands, that is, those ligands that bind protein, are isolated and identified, for example, by mass spectroscopy or NMR, preferably directly from the binding complex, for example, “on bead.” Protein(s) bound to identified active ligand(s) are identified, preferably from the same binding complex, for example, by mass spectroscopy, peptide sequencing, or other known processes. Alternatively, an identified active ligand can be used to isolate its specific binding protein receptor. The isolated protein receptor is then identified, for example, by sequence analysis or other known methods.

In one particular embodiment of the invention, a ligand library is incubated with two or more differentially labeled protein mixtures, for example, obtained from two or more different protein sources, such as a normal set of proteins obtained from normal tissue and an abnormal set of proteins obtained from diseased tissue. The protein sets are preferably mixed, and then incubated with a ligand library. Ligand-protein complexes are isolated, for example, according to the specific protein labels. Ligands that selectively and/or differentially bind with one set of proteins are identified. The protein(s) binding to these selective ligands can be identified from the same binding complex, for example, on a single resin bead. Alternatively, the identified selective ligands can be used to isolate the corresponding binding protein(s) that are then identified.

The inventive process provides a rapid and efficient identification of specific members of a previously unknown ligand-protein binding pair. The process can be readily automated, providing greater efficiencies. In a most preferred embodiment, efficiencies are achieved by carrying out multiple process steps using the same reactants, for example, synthesizing the ligand library directly onto a solid support that is then used for incubating the ligand with the protein mixture; detecting the specific ligand-protein binding pairs while immobilized on the same solid support, and identifying each of the ligand and protein from the same immobilized binding complex. Accordingly, the process of the invention eliminates transfers, reactions, purification, and other steps that reduce efficiency, and otherwise impede in the discovery of ligand-binding interactions. Using the process of the invention, novel ligand-protein binding pairs are efficiently detected and identified, and provided as drug leads and targets.

### BRIEF DESCRIPTION OF THE DRAWINGS

The invention may be more completely understood with reference to the following detailed description of various embodiments of the invention and specific working Examples in connection with the accompanying Figures, in which:

FIG. 1 is a schematic representation of one embodiment of the process invention, showing a ligand library incubated with a single set of proteins to identify specific ligand-protein binding pairs.

FIG. 2 is a schematic representation of one embodiment of the process invention, showing a ligand library incubated with two or more different sets of proteins to identify specific and selective differential ligand-protein binding pairs.

### DETAILED DESCRIPTION OF THE INVENTION

Definitions:

As used herein, the following words are intended to have the specified definitions:

Affinity probe refers to a detection probe that uses a binding (affinity) interaction as part of the detection process, for example biotin-avidin, antibody-antigen and the like.

Detection probe refers to a compound, generally a small molecule, peptide or protein, polynucleotide, and the like, that is used for detecting a binding interaction, such as ligand binding to protein. The detection probe may produce a detectable signal, such as color, fluorescence, and the like, or may react with a known probe, such as an affinity probe that provides the detection signal.

Immobilized as used herein, means that a molecular entity is covalently attached to a solid support.

Low abundance proteins refers to proteins present in low amounts in a protein sample so as to be masked by other proteins in typtical detection methods, and include, for example, transcription factors, protein kinases, and phosphatases.

Library refers to a collection of molecular entities obtained after a series of one or more synthetic transformations.

Ligand refers herein to a molecule that binds to a biological macromolecule, for example a protein and the like.

Linker refers herein to a molecular entity that can be used to bind a ligand to a solid support. Preferred in this invention are molecular entities that can be specifically cleaved. Examples include acid labile (Rink amide), base labile (HMBA), photolabile (2-nitrobenzyl and 2 nitrovaleryl), other specific cleavage entities (allyl, silyl, safety catch sulfonamide), and the like.

Parallel Array refers to a collection of molecular entities in a ligand library generated by parallel synthesis.

Peptidomimetic refers to non-peptide molecules that mimic the binding characteristics of peptides.

Photoprotein refers to a protein that emits fluorescence or chemoluminescence, for example green flourescent protein (GFP) or luciferase.

Previously unknown protein-ligand binding pair refers to a protein and ligand that are found to bind to each other through the implementation of this process but that specific ligand and protein binding interaction was not known before.

Protein mixture refers to a solution of proteins that have been isolated from characterized cell cultures, specific cells, cells from a whole organism or tissue, mixtures of cells from normal and/or diseased tissue, and the like.

Protein receptor or receptor refers to a protein that binds to a ligand, and includes, for example, surface receptors, enzymes such as proteases, protein kinases, phosphatases, and the like, transcription factors, co-factors, adaptor proteins, structural proteins, and the like.

Small organic molecules or compounds refer herein to non-oligomeric compounds produced by chemical synthesis and generally having a size of less than 600 microns (mu).

The present invention provides a process for identifying the structure of previously unknown members of specific ligand and protein binding pairs. The invention provides a process using libraries of compositionally defined, spatially separated, yet structurally unknown ligands to isolate protein receptors from protein mixtures by virtue of specific binding affinity. In one embodiment, using the process and tools of the invention, specific proteins characteristic of biological processes and their matching binding ligands are simultaneously identified. In particular, the invention provides a novel process for identifying particular proteins as potential drug targets together with a matched potential drug lead (ligand).

As used herein, the term “ligand” refers to a molecule that binds to a protein. The identified members of a ligand-protein binding pair are useful as potential drug targets and lead compounds. Furthermore, the protein-ligand complexes isolated and identified by the process invention are also useful to aid in mapping out biological pathways and pointing to functions of the identified protein. The process invention provides preliminary information on potential combination drug therapy as well as toxicology of the drug.

This process invention provides significant advantages over alternative processes of drug discovery by virtue of its ease, speed, broad generality and applicability, and yields a large amount of information in a short time. Furthermore, as demonstrated in the Examples below (for example, Examples 32 and 33), the process of the invention provides matching ligand-protein pairs for low abundance proteins, hydrophobic proteins, and membrane proteins (for example, G-coupled protein receptors) that are typically difficult to isolate and match with a binding partner.

The Solid Phase Library

In the present invention, libraries of compounds are used to screen biological mixtures. As used herein, the term “library” means a collection of molecular entities obtained after a series of reactions. In one embodiment, these molecular entities can be natural oligomers (occurring in Nature) such as peptides, glycopeptides, lipopeptides, nucleic acids (DNA or RNA), or oligosaccharides. In another embodiment, they can be unnatural oligomers (not occurring in Nature) such as chemically modified peptides, glycopeptides, nucleic acids (DNA or RNA), or, oligosaccharides, and the like. For examples and recent reviews see: Graven et al., 2001, J. Comb. Chem., 3: 441-52; St. Hilaire et al., 2000, Angew. Chem. Int. Ed. Engl., 39: 1162-79; James, 2001, Curr. Opin. Pharmacol., 1: 540-6; Marcaurelle et al., 2002, Curr. Opin. Chem. Biol., 6: 289-96; Breinbauer et al., 2002, Angew. Chem. Int. Ed. Engl., 41: 2879-90. The chemical modification may be, for example, the use of unnatural building blocks while maintaining the natural bond linking the units (for example, peptide/amide as shown in Example 5), the use of natural building blocks with modified linking units (for example, oligoureas as discussed in Boeijen et al, 2001, J. Org. Chem., 66: 8454-8462; oligosulfonamides as discussed in Monnee et al, 2000, Tetrahedron Lett., 41: 7991-95), or combinations of these (for example, statine amides as discussed in Dolle et al, 2000, J. Comb. Chem., 2: 716-31.). In yet another embodiment, the molecular entities may comprise non-oligomeric molecules such a peptidomimetics or other small organic molecules. Peptidomimetics are compounds that mimic the action of a peptidic messenger, such as bicyclic thiazolidine lactam peptidomimetics of L-proplyl-L-leucyl-glycinamide (Khalil et al, 1999, J. Med. Chem., 42: 2977-87). Small organic molecules are non-oligomeric compounds of less than about 600 mu containing any of a variety of possible functional groups that are the product of chemical synthesis, or isolated from nature, or isolated from nature and then chemically modified, and include, for example, Bayer's urea-based kinase inhibitors (Smith et al., 2001, Bioorg. Med. Chem. Lett., 11: 2775-78). For reviews containing pertinent examples see: Thompson et al., 1996, Chem. Rev., 96: 555-600; Al-Obeidi et al., 1998, Mol. Biotechnol., 9: 205-23; Nefzi et al., 2001, Biopolymers, 60: 212-9; Dolle, 2002, J. Comb. Chem., 4: 369-418.

Selection of the ligand library is dependent upon the desired screening and identification desired. For example, the process invention can utilize a totally random library designed to contain interesting and greatly diverse compounds. An advantage of this approach is that the outcome of the screening is not prejudiced in any specific manner. Since the process invention permits screening of millions of diverse compounds, for example, immobilized in 10 g of resin, a large number of random molecules can be used in the ligand library.

Alternatively, a smaller, targeted library (hundreds to thousands of compounds) can be used, for example, starting with a known compound or compounds, and providing numerous variations of these known compounds for targeted screening for new ligand-protein binding pairs. The smaller, targeted library can also comprise random molecules.

The library may contain a parallel array of random modifications of one or more ligands. In one embodiment, the library may be formed as a parallel array of random modifications to a known compound or compounds.

Solid Support

In this invention, the compounds of the library are preferably bound to a solid support, conferring the advantage of compartmentalized “mini-reaction vessels” for the binding of proteins with an optimal ligand(s). The solid support can be, for example, a polymer bead, thread, pin, sheet, membrane, silicon wafer, or a grafted polymer unit; for example, a Lantern™ (Mimotopes™, found at the website mimotopes.com under combichem/lanterns.html). Each member of the library is a unique compound and is physically separated in space from the other compounds in the library. Depending on the mode of library synthesis, each library member may contain, in addition, fragments of the library member. Since ease and speed are important features of this process invention, it is preferred that the screening (incubating) step take place on the same solid support used for synthesis of the library, and also that identification of the members of the binding pair can take place on the same support, such as on a single resin bead. Thus, solid supports useful in the process invention satisfy the criteria of not only being suitable for organic synthesis, but are also suitable for screening procedures, such as “on-bead” screening as described in the Examples below. Hydrophilic supports described below are useful supports. Screening of libraries and ligands with purified individual proteins or cells has been attempted on individual resin beads such as TentaGel (commercially available from Rapp polymere, Tübingen, Germany), ArgoGel (commercially available from Argonaut Technologies Inc., San Carlos, Calif.), PEGA (commercially available from Polymer Laboratories, Amherst, Mass.), POEPOP (Renil et al., 1996, Tetrahedron Lett., 37: 6185-88) and SPOCC (Rademann et al, 1999, J. Am. Chem. Soc., 121: 5459-66). Examples of on-bead screening attempts are described in the following references: Chen et al., 1996, Methods Enzymol., 267: 211-19; Leon et al., 1998, Bioorg. Med. Chem. Lett., 8: 2997-3002; St. Hilaire et al., 1999, J. Comb. Chem., 1: 509-23; Smith et al., 1999, J. Comb. Chem., 1: 326-32; Graven et al., 2001, J. Comb. Chem. 3: 441-52; Park et al., 2002, Lett. Peptide Sci., 8: 171-78). TentaGel and ArgoGel are made up of polyethylene chains grafted on to a polystyrene core. However, use of these supports in biological screening is limited by a size restriction, and by denaturation of certain proteins, particularly enzymes. Conversely, PEGA (PolyEthyleneGlycol Acrylamide copolymer; Meldal M., 1992, Tetrahedron Lett., 33: 3077-80), POEPOP (PolyOxyEthylene-PolyOxyPropylene; Renil et al., 1996, Tetrahedron Lett., 37: 6185-88) and SPOCC (Super Permeable Organic Combinatorial Chemistry; Rademann et al, 1999, J. Am. Chem. Soc., 121: 5459-66) resins are made primarily of polyethylene glycol and swell well in organic as well as aqueous solvents. Because they have very reduced or no non-specific binding, PEGA and SPOCC resins have been effectively used in the screening of myriad proteins including enzymes of different classes. Furthermore, these resins are available in different pore sizes and can allow large proteins to enter while retaining activity. For example, PEGA6000 resins allow proteins up to 600 kDa to enter. In the Examples below, PEGA4000 and PEGA1900 resin with a molecular weight cut off of 200 and 90 kDa, respectively, were used for screening. In principle, any hydrophilic support that is useful for compartmentalized synthesis, retains the activity of the proteins, and has minimal non-specific binding, may be used in this process invention.

Ligand Library Synthesis

The ligand library may be synthesized by known processes, for example, by parallel synthesis giving rise to small libraries (10 to 1000 members) (for a recent review see: Dolle et al., 2002, J. Comb. Chem., 4: 369-418), or by split/mix or split and combine methodology, as described, for example, in Furka et al., 1991, Int. J. Peptide Protein Res., 37:487-493 and Lam et al., 1991, Nature, 354: 82-84. The split/mix or split and combine method is a preferred method for generating a large library, due to the exponential increase in the number of varied compounds produced. The split/mix method gives rise to a one-bead-one-compound library of large size (1000 to millions of members). In this invention, the one-bead-one-compound library is preferred, and is demonstrated in the Examples below.

The ligand library members may be built up by performing all compound forming reactions directly on a solid phase. Alternatively, the ligand library members can be prepared by linking together preformed building blocks on a solid phase. The resulting library members can be small organic molecules or oligomeric compounds. In both cases, the molecules contain a variety of functional groups. The functional groups can be, for example, alkynes, aldehydes, amides, amines, carbamates, carboxylates, esters, hydroxyls, ketones, thiols, ureas, and the like. The small organic molecule can belong to various classes of compounds, including but not limited to, heterocycles (for example, hydantoins, benzodiazepines, pyrrolydines, isoquinolines), carbocyclic compounds, steroids, nucleotides, alkaloids, and lipids (for reviews containing examples see: Thompson et al., 1996, Chem. Rev., 96: 555-600; Al-Obeidi et al., 1998, Mol. Biotechnol., 9: 205-23; Nefzi et al., 2001, Biopolymers, 60: 212-9; Nicolau et al., 2001, Biopolymers, 60: 171-193; Dolle, 2002, J. Comb. Chem., 4: 369-418).

Where the ligand library members are oligomeric, as demonstrated in the Examples below, the building blocks may be selected from a wide repertoire of suitably protected bi- or tri-functional compounds, for example, amino acids, sulfonic acids, aliphatic acids, aromatic acids, glycosyl amino acids, lipidyl amino acids, heterocyclic amino acids, haloamines, aminohydroxy compounds, diamines, and azido acids. The building blocks may be connected using various types of chemical bonds, for example, an amide, a thioamide, an amine, a sulfonamide, a urea, a thiourea, an ether, a thioether, an ester, a sulfate, a phosphate, a phosphine, a carbonate, a —C—C-bond, a double bond, a triple bond, or a silane. The oligomer may be linked using only one type of chemical bond or using a mixture of bonds. When the library members are amino acids, they preferably are molecules containing about 2 to 40 amino acids. More preferred are molecules of about 3 to 20 amino acids, and most preferred have about 3 to 12 amino acids. For example, molecules of 3, 4, 5, 6, 7, 8, 9, 10, 11, or 12 amino acids work well in the ligand library.

The ligand library members may be directly attached to a solid support or indirectly attached via a variety of linkers, preferably by covalent bonds (For reviews describing linkers for solid phase synthesis, see: Backes et al., 1997, Curr. Opin. Chem. Biol., 1: 86-93; Gordon et al., 1999, J. Chem. Technol. Biotechnol., 74: 835-851). The linkers may be acid labile (for example, the Rink amide as described in Rink, 1987, Tetrahedrom Lett., 28: 387 and traceless silyl linkers as described in Plunkett et al., 1995, J. Org. Chem., 60: 6006-7), base labile (for example, HMBA as described in Atherton et al. 1981, J. Chem. Soc. Perkin Trans, 1: 538), or photolabile (for example, 2-nitrobenzyl type as described in Homles et al., 1995, J. Org. Chem., 60: 2318-2319). The linkers may be more specific and restrictive of the type of chemistry performed, such as silyl linkers (for example, those cleaved with fluoride as described in Boehm et al., 1996, J. Org. Chem., 62: 6498-99), allyl linkers (for example, Kunz et al., 1988, Angew. Chem. Int. Ed. Engl., 27: 711-713), and the safety catch sulfonamide linker (for example, as described in Kenner et al., 1971, Chem. Commun., 12: 636-7). In the Examples below, the invention is illustrated by the use of a photolabile linker, 2-nitrovaleryl (1) as described in Homles et al., 1995, Supra, that proves very robust to a wide variety of chemistries and hastens the identification process.

In some embodiments a spacer molecule may be used. When used, the spacer molecule can be a peptide or non-peptide molecule and does not interact with most or all proteins, and thereby does not interfere in the screening process. Such spacers are useful for aiding the identification of the ligand by MALDI-TOF-MS.

Screening Processes

In the process invention, biological material is isolated and protein mixtures obtained for screening of the ligand libraries. The proteins can be obtained from any source, including, for example, simple organisms such as fungi, viruses, protozoans and bacteria to more complex organisms such as plants and animals, including mammals and particularly, humans. The biological material may be extracted from individual cell lines, (illustrated in the Examples with myocytes), from cellular organisms (illustrated in the Examples with E. coli), or from tissue containing a large variety of cell types. Protein mixtures can also be obtained from cellular systems expressing a cDNA library that may be tagged, for example, with a genetic label that is co-expressed and used for detection analysis. Suitable genetic tags include, for example, myc and photoproteins such as Green Fluorescent Protein (GFP).

The intricacy of the extraction procedures increases with the complexity of the source of the biological material. There are various known ways of isolating proteins from cells, tissue, and organisms while preserving the activity of the protein. The proteins can be extracted and solubilized using a variety of auxiliary substances such as detergents and ureas. This extraction procedure is particularly important for larger, hydrophobic proteins such as membrane proteins. The use of detergents, ureas, and salt is compatible with screening on solid phase resins in contrast to methods using 2-D gels. Proteins can be extracted using standard equipment such as the French Press and sonicator. The extraction procedure can be manipulated to enrich for low abundance proteins or to isolate a particular class of proteins. General protocols for the extraction of proteins from different organisms are readily available. See, for example, 2-D Proteome Analysis Protocols, A. J. Link (Ed), 1^{st} Ed, 1999, Humana Press: Totowa)

Detecting

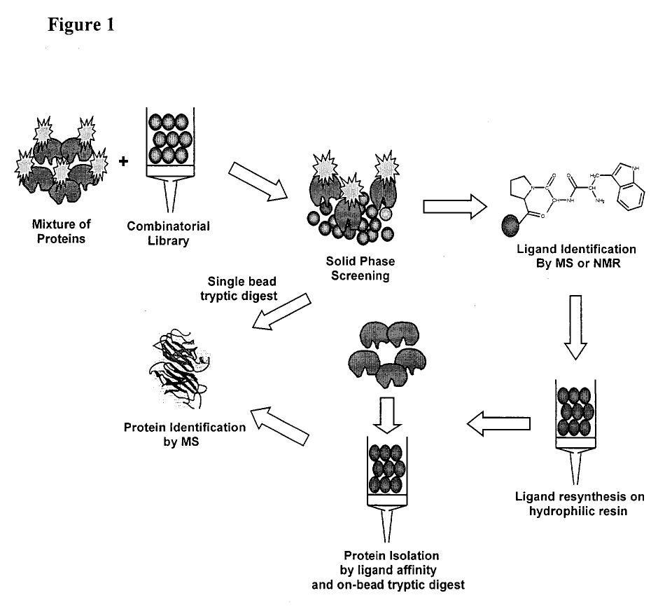

A variety of suitable methods are useful for detecting the ligand-protein binding pairs. For example, where a single protein mixture is used (see FIG. 1), the extracted protein may be immediately incubated with the immobilized ligand library, and, after washing, bound protein can be detected directly in the binding complex by the application of a detection molecule to the incubation mixture, such as silver or fluorescent dye that does not interact with the ligand or the solid support. In another embodiment, the mixture of proteins may be labeled, for example, with a fluorescent dye such as Oregon Green 514 (green; See Example 11), anthranilic acid (blue; See Example 13), Rhodamine red (red; See Example 11), or other commonly used fluorescent probes. The detection probe can also be a probe that produces chemoluminescence, such as luciferase or aequorin. See for example, world wide web (WWW) sites “probes.com” and “amershambiosciences.com/aptrix/upp00919.nsf/Content/DrugScr+CyDye+Fluors+introduction” for a description of cyanine fluorescent dyes. In these embodiments, after incubation of ligands with proteins, the library is washed and ligand-protein binding complexes will be detected via the label, for example, fluorescence or color. These ligand-protein binding pairs can be immediately isolated using automatic or manual sorting procedures.

Similar labeling procedures can also be applied to the identification of differential matched ligand-protein binding pairs from multiple, related sources (see Example 11). For example, as taught in Example 11, a mixture of proteins from normal tissue and a mixture of proteins from diseased tissue can be differentially labeled, a different dye or fluorescent label (and the like) for each of the protein mixtures. After incubation with the ligand library and washing away unbound protein, the differential protein-ligand binding pairs, those that demonstrate selectivity, that is, are specific to one set of proteins, are detected and isolated automatically or manually on the basis of the particular label or detection probe.

In another embodiment, proteins are labeled with an affinity probe (tag) such as biotin. After incubation and washing of the proteins and ligand library to remove unbound protein, ligands bound with tagged, for example, biotinylated proteins may be detected, for example, using streptavidin complexed with a phosphatase or a peroxidase. After addition of a suitable phosphatase or peroxidase substrate, the ligand-protein binding complex is detected.

In another embodiment, proteins bound to ligands can be detected using radioactivity. In this case, the organism or cell is fed with a radioactive amino acid that is incorporated into its proteins. After incubation of the radioactive proteins with the ligand library and washing, bound radioactive protein-ligand is detected by, for example, autoradiography, and the protein-ligand binding pairs are isolated.

In yet another embodiment, particular classes of proteins that bind to ligands can be detected using specific probes, for example, a family-specific antibody in an immunoassay such as an ELISA assay. Treatment with a conjugated monoclonal antibody for a family of proteins after incubation and washing, for example, provides information about the expression of related proteins. Where the protein mixtures are obtained from related proteins sources, for example, from diseased and normal tissue, the ligand libraries can be incubated separately with each set of proteins. After detection and identification of the ligand-protein binding pairs, an assessment of the expression of the particular protein class in each state (for example, normal vs. diseased) can be determined. A monoclonal antibody may be conjugated to a fluorescent dye or to an enzyme such as peroxidase or alkaline phosphatase for quantification by ELISA. The antibody may also be conjugated to ferro-magnetic beads by known, routine techniques. The magnetic beads concentrate near the location of the protein forming a “rosette” around solid support beads, or on the membrane sheet, or thread for detection.

In a final embodiment, for example, where a single protein mixture is used, the extracted protein may be immediately incubated with the immobilized ligand library, and, after washing, detection of bound protein and isolation of the specific ligand-protein binding pair is done without any labelling, for example by measuring refractive index changes of the resin beads. Beads containing both proteins and ligand will have a different refractive index than beads containing only ligand. The refractive index changes could be detected from the light scattering when using an automated bead sorter as described below; or by using a custom-made instrument based on the principles of surface plasmon resonance or (SPR).

Isolation

Bound protein-ligand complexes or pairs can be isolated from the bulk of the ligand library by various means, including, for example, manually sorting beads containing bound labeled protein with the aid of a microscope, sorting by fluorescence or by color depending on the screening process used. Alternatively, the sorting process may be automated with the use of specially designed, commercially available bead sorters (Union Biometrica, Sommerville, Mass.) and detecting fluorescence intensity (Meldal, 2002, Biopolymers, 66: 93-100). In general, resin beads can be sorted at a rate of about 100 beads per second, or even faster depending on the equipment used and its reading capacity. A range of about 5-30 beads per second is generally used with known instruments. Slower rates may be used to increase reading accuracy. Preferred, for example, is a rate where only one resin bead passes thru through the detector at a time.

Identification of Ligands

After detection and isolation of the protein-ligand complexes, the ligand can be identified. The process for identification of the ligand depends on the type of library used. For a library of primarily oligomeric compounds, the complexed ligand can be analyzed by Mass Spectroscopy (MS), particularly if the library was synthesized in such a way that the synthetic history of the compound is captured, for example, using a capping procedure to generate fragments of the compound that differ in mass by one building block (see, for example, Youngquist et al., 1995, J. Am Chem. Soc., 117: 3900-06). This capping procedure is most efficient when the cap and the building block are reacted at the same time. The capping agent can be any class of compound that has at least one functional group in common with the building block used to generate the oligomer, so that both the capping agent and the building block can react when added to the resin in an appropriate ratio. Alternatively, the capping agent can have two functional groups in common with the building block where one of the groups in common, such as the group in the building block that is used for the elongation of the oligomer, is orthogonally protected. For example, in a synthesis of a peptide using the Fmoc strategy shown in the examples below, the capping agent could be the same as the building block but with a Boc group protecting the reactive amine instead of the Fmoc group (see Examples 5, 6, and 7 and St. Hilaire et al., 1998, J. Am. Chem. Soc., 120: 13312-13320). In another example, if the building block is a protected haloamine, the capping agent could be the corresponding alkylhalide.

Where the ligand library is synthesized by parallel synthesis (a parallel array), the binding ligand can be identified simply by the knowledge of what specific reaction components were reacted in a particular compartment. The structure can be confirmed by cleavage of a small portion of compound from the solid support and analyzed using routine analytical chemistry methods such as infrared (IR), nuclear magnetic resonance (NMR), mass spectroscopy (MS), and elemental analysis. For a description of various analytical methods useful in combinatorial chemistry, see: Fitch, 1998-99, Mol. Divers., 4: 39-45; and Analytical Techniques in Combinatorial Chemistry, M. E. Swartz (Ed), 2000, Marcel Dekker: New York.

In the case of libraries synthesized by the split-mix approach where the precise structure of the compound is unknown, the complexed ligand can be identified using a variety of methods. The compound may be cleaved off the solid support, for example, resin bead, and then analyzed using IR, MS, or NMR. For NMR analysis, larger beads containing approximately 5 nmoles of material can be used for the acquisition of 1-dimensional (1-D) and 2-dimensional (2-D) NMR spectra. Furthermore, these spectra can be attained using high-resolution MAS NMR techniques. Alternatively, high resolution-MAS NMR spectra can be acquired while the ligand is still bound to the solid support, as described for example, in Gotfredsen et al., 2000, J. Chem. Soc., Perkin Trans., 1: 1167-71.

Typically, resin beads used for library synthesis contain about 100 to 500 pmoles of material, which is generally insufficient for direct analysis using NMR techniques. In such situations, the ligand libraries can be synthesized with special encoding to facilitate identification of the ligand For a review of encoding strategies employed in combinatorial chemistry see: Barnes et al., 2000, Curr. Opin. Chem. Biol., 4: 346-50. Most coding strategies include the parallel synthesis of the encoding molecule (for example, DNA, PNA, or peptide) along with the library compounds. This strategy is not preferred, as it requires a well-planned, time consuming, orthogonal protecting group scheme. Furthermore, the encoding molecule itself can sometimes interact with the protein receptor leading to false positives. Alternatively, the ligand library members can be encoded using radiofrequency tags. This method alleviates the problem of false positives stemming from the coding tags, but is generally only useful for small ligand libraries in the one-bead-one-compound system due to the sheer bulk of the radiofrequency tag. Alternatively, single beads can be analyzed in a non-destructive manner using infrared imaging. However, this method gives limited information and while useful for pre-screening, is not recommended for conclusive structural determination. MS can be used alone to identify the ligand library member. The ligand can be cleaved from the solid support, the molecular mass determined, and subsequently fragmented into sub-species to conclusively determine the structure. MS-based methods of ligand identification are useful in this invention, as they require very little material, and can utilize pico- to femtomole amounts of compound.

Process for Isolation and Identification of Binding Protein Member

Once the binding ligand member has been identified and isolated with its bound protein, the binding protein member can be identified. In one embodiment, a resin bead containing the binding pair is cut into two portions. One portion of the bead is used to identify the ligand, while the other portion is used to identify the protein. This can be accomplished, for example, by performing systematic degradation of the protein on-bead. Most often, the protein can be broken down into its constituent peptides enzymatically, for example using trypsin or other known peptidases. General protocols for enzymatic breakdown of proteins during proteomic analysis can be found, for example, in 2-D Proteome Analysis Protocols, A. J. Link (Ed), 1^{st} Ed, 1999, Humana Pr: Totowa. Given the hydrophilic nature of the resins, trypsin works efficiently on-bead, and can efficiently cleave native proteins as well as proteins that have been covalently modified with a detection probe. The number of reasonably sized peptides generated by enzymatic cleavage is improved if the proteins are first denatured. Denaturation is easily accomplished on-bead, for example, on PEG-based resins that are robust and solvated in most denaturants used, such as guanidine HCl and urea. Other cleavage enzymes may be used, for example, endoprotease Arg-C, endoprotease Lys-C, chymotrypsin, endoprotease Asp-N, and endoprotease Glu-C. Sometimes, chemicals such as CNBr (as described, for example, in Compagnini et al., 2001, Proteomics, 1: 967-74) and [cis-Pd(en)(H2O)₂]²⁺ (as described, for example, in Milovic et al., 2002, J. Am. Chem. Soc., 124: 4759-69) may be used to degrade a protein into its constituent peptides.

Alternatively, the identified ligand can be resynthesized and coupled to an affinity support such as sepharose or sephacryl, and the protein member purified by affinity chromatography. Unlabelled protein mixture is applied to the affinity column and, after washing of the unbound protein, bound protein is eluted with solubilized ligand. This route is time and reagent consuming. The ligand must first be synthesized and purified, and then attached to the affinity support. It should also be produced in sufficient quantities that the required concentration can be used to elute protein from the affinity column. Alternatively, buffers of different pH and high salt can be used to elute protein. It can sometimes be difficult to elute multimeric proteins from affinity columns using a monovalent ligand because of avidity effects.

To expedite the process and alleviate the aforementioned problems, the protein can be degraded into peptides while still bound to its ligand-binding partner, and the generated peptides analyzed. For example, the ligand is resynthesized on small scale (25-50 beads) on PEGA4000 resin, the same resin used for library synthesis. After binding of unlabelled protein from the mixture and washing off the unbound protein, the protein-ligand complex can be immediately degraded into the constituent peptides either enzymatically or chemically, using known processes and reagents and the peptides analyzed, for example, by peptide mass fingerprinting, or other known methods. Using this process several ligand-protein complexes can rapidly be digested. This process can be readily automated.

The protein bound to the ligand can be identified by MS or Edman degradation sequencing. For general protocols on the identification of proteins using proteomics techniques, see, for example, 2-D Proteome Analysis Protocols, A. J. Link (Ed), 1^{st} Ed, 1999, Humana Pr: Totowa. Protein can be identified from its peptide mass fingerprint, for example, using the mass of some of the constituent peptides obtained from enzymatic digests. The mass of the mixture of peptides generated from the digested proteins can be determined using MALDI-TOF-MS or ES-MS. The peptide masses or fingerprints are used to search databases of known proteins and gene products to identify the protein(s). To increase accuracy of the protein identification in the absence of other limiting information such as pI and mass, the results of several digests using different processes for cleavage are combined. Instead of, or in addition to, generating peptide fingerprints, a single peptide from the protein can be fragmented, and its amino acid sequence determined. The sequence can be used to identify known and unknown proteins, for example, by comparing to protein databases. The use of MS to identify the proteins(s) is well suited to the degradation of protein complexes on single beads, since very little material is required for identification (pico-femtomole). Alternatively, proteins can be identified using N-terminal sequencing via Edman degradation; provided that the N-terminus is not blocked. This generally requires larger quantities of material (picomole).

Advantages of the Invention

The high throughput process invention described herein provides several advantages over known processes for drug discovery. Among these advantages are increased speed and accuracy in the simultaneous identification of a ligand molecule and its matched protein-binding partner.

The process of the invention can be accomplished on a solid support, preferably on resin beads, and permits synthesis, screening, isolation, and identification of ligand and protein steps to be quickly and efficiently processed on a single bead. The process can be readily automated, for example, using known automatic systems for synthesis, incubation, isolation, and identification steps, such as robotic systems for cleavage of ligand, spotting on to MS targets, adding enzymes for protein digestion, and the like. Using Mass Spectrometry, MALDI, and NMR as described in the Examples, each of the ligand and protein can be identified “on bead.”

The process invention also provides an advantage due to the large diversity of libraries that can be used, for example, in excess of 10,000 different compounds. Virtually millions of compounds can be rapidly screened, for example, in resin systems employing one compound per bead, for example, using about 3 to 5 million beads available in about 10 g of resin.

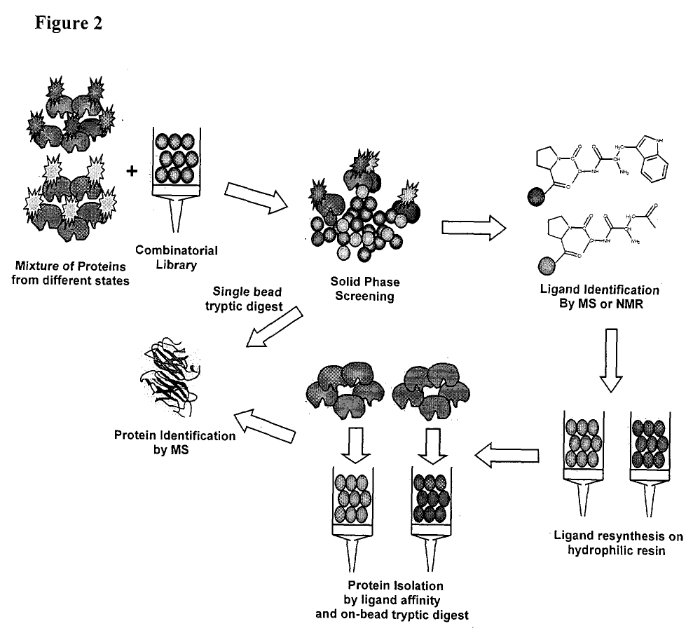

Using the claimed process, it is possible to rapidly identify ligand protein binding complexes that are “drugable,” that is, to identify useful ligands that bind precise proteins and to avoid non-useful ligands. The process further enables the rapid identification of families of proteins and/or non-related proteins that bind to the same or similar ligands. Such information delineates potential selectivity of a drug candidate and provides preliminary toxicological information to aid the drug selection process. The process invention further provides identification of classes of ligands that bind a particular protein increasing the number of “hits” that can be developed into lead compounds for a particular protein target. When the process invention is carried out in the differential manner as described in FIG. 2, that is, using differentially labeled proteins, for example, from a normal and diseased tissue, all the above features are added to the determination of selective ligand/protein pairs that can be used for diagnostic and therapeutic product development.

In sum, one great advantage of the claimed invention is the ability to take a very large number of unknown ligands and/or unknown proteins, and in a very short time and efficient manner identify particular, previously unknown ligand/protein binding pairs that are identified, matched, and characterized as described above.

### EXAMPLES

The invention may be better understood with reference to the following Examples. These Examples illustrate the invention, and are not intended to limit its scope.

In the Examples below, the synthesis of ligand libraries and use of these libraires in the process invention is exemplified. The chemical entities used in the exemplified synthesis are shown in the tables and schemes below, with identification numbers in parentheses (x).

In the tables and schemes shown below, the compound numbers (x) are denoted “a” where R₁ is Fmoc and “b” where R₁ is Boc.

TABLE 1
Linker, spacer, and genetically encoded (natural) amino acid building blocks
used in the synthesis of Libraries 1, 2, 3, 4, and 5

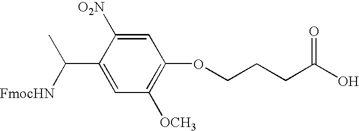

Pll (1)

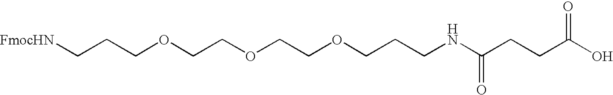

(2)

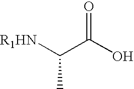

Ala (3)

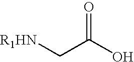

Gly (4)

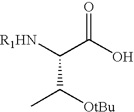

Thr (5)

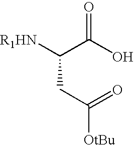

Asp (6)

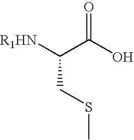

Met (7)

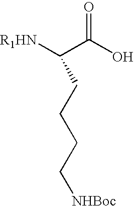

Lys (8)

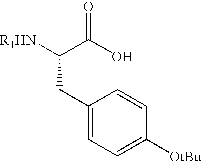

Tyr (9)

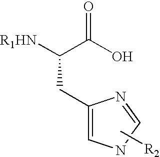

His (10)

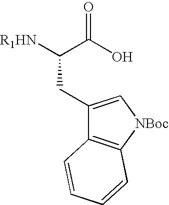

Trp (11)

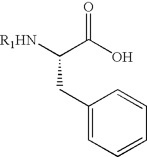

Phe (12)

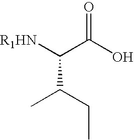

Ile (13)

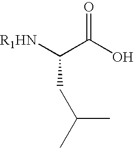

Leu (14)

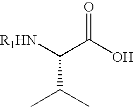

Val (15)

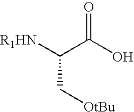

Ser (16)

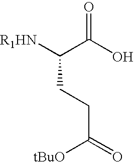

Glu (17)

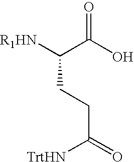

Gln (18)

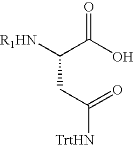

Asn (19)

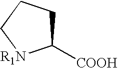

Pro (20)
R₁ = Fmoc, Boc
For 10, R₂ = Boc

TABLE 2
Aliphatic encoding tags and other building blocks
used in the synthesis of Libraries 1, 2, 3, 4, and 5

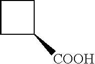

Aze (21)

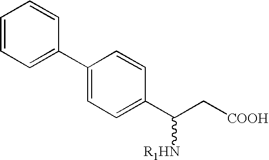

Abi (22)

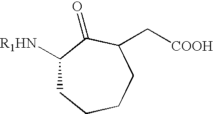

Acc (23)

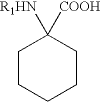

Che (24)

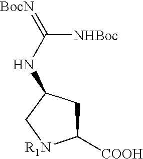

Gua (25)

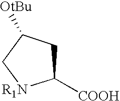

Hyp (26)

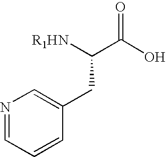

Pya (27)

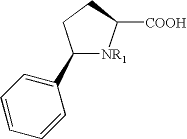

Ppy (28)

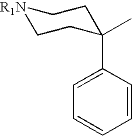

Pip (29)

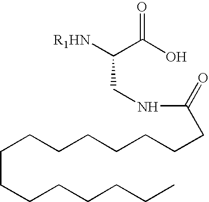

Pal (30)

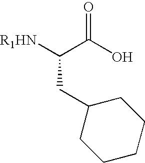

Cha (31)

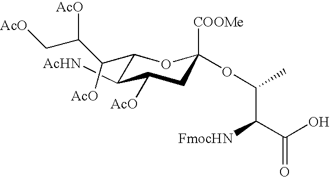

T(Sa) (32)

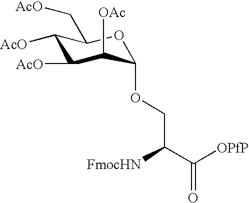

ManS (33)

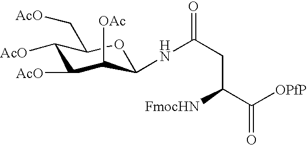

ManN (34)

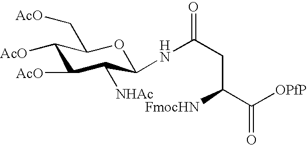

GlcNN (35)

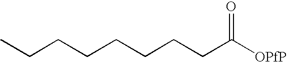

(36)

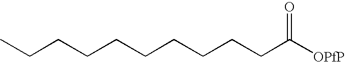

(37)

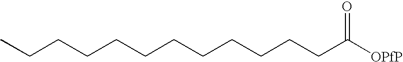

(38)
R₁ = Fmoc, Boc
(a) (b)

TABLE 3
Compounds Used for Synthesis of Library 4 and 5.

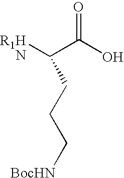

Om (39)

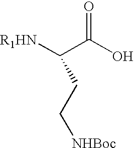

Dab (40)

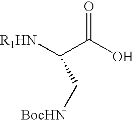

Dap (41)

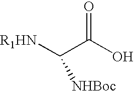

Agly (42)

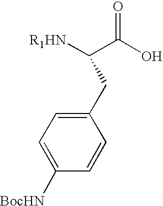

Aphe (43)

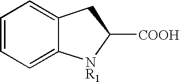

Ind (44)

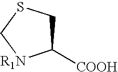

Tpro (45)

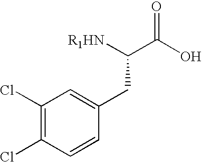

CPhe (46)

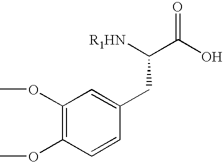

MPhe (47)

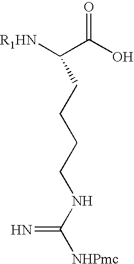

Arg (48)

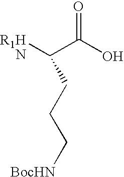

(49)

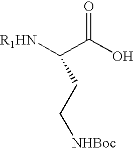

(50)

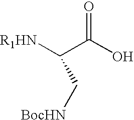

(51)

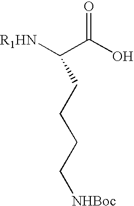

(52)

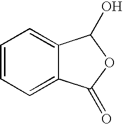

Cba (53)

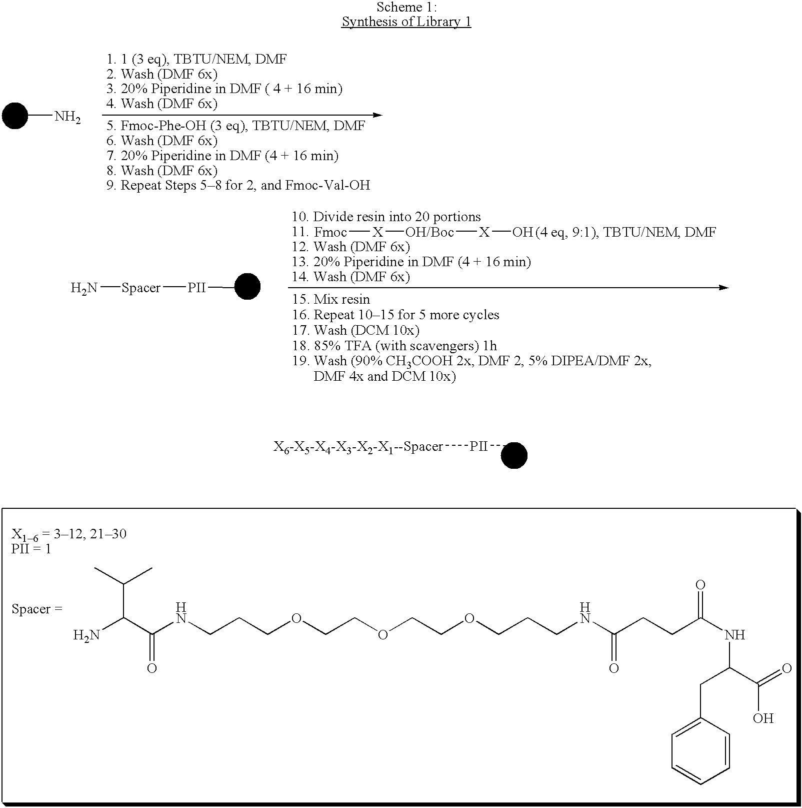

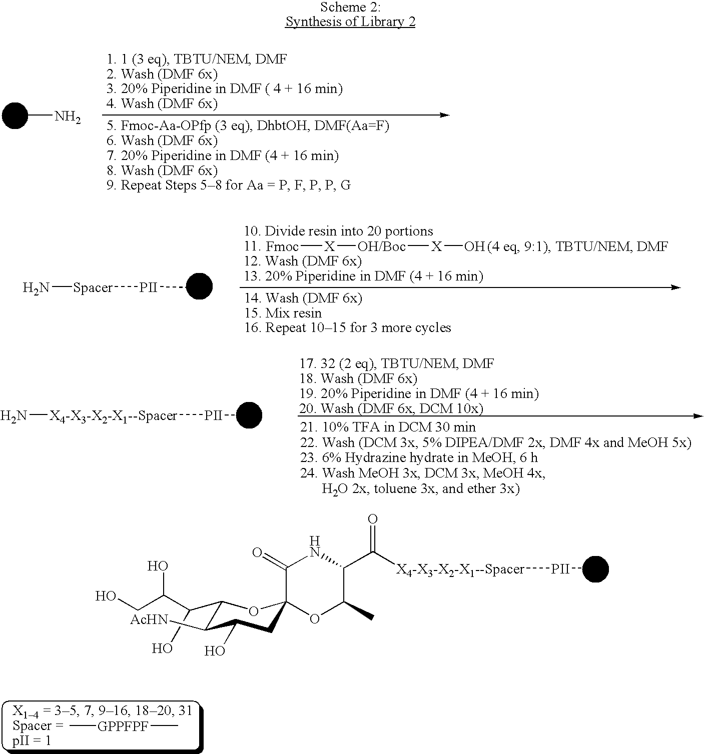

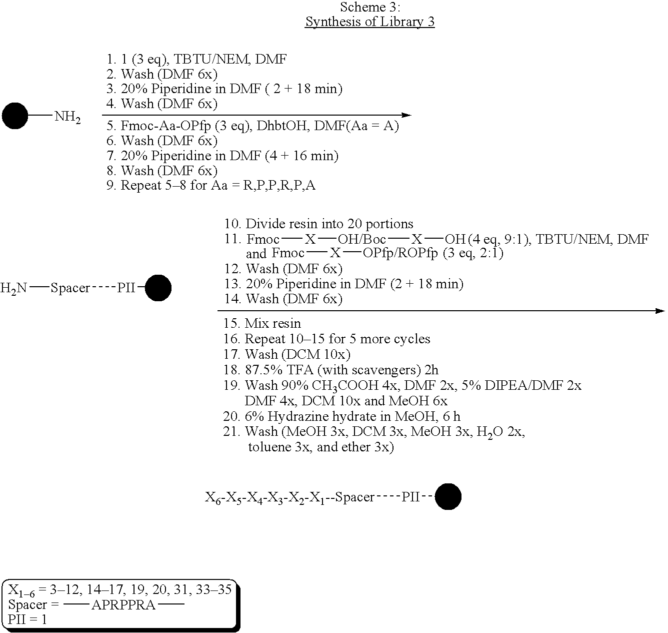

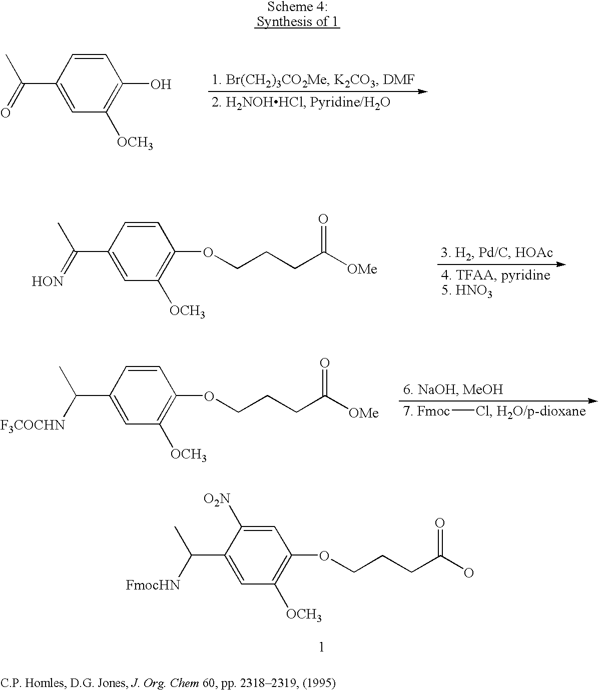

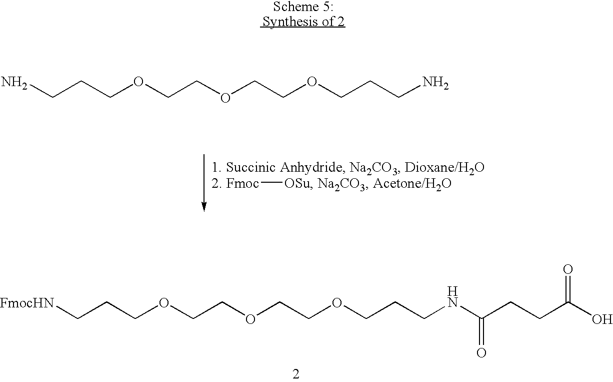

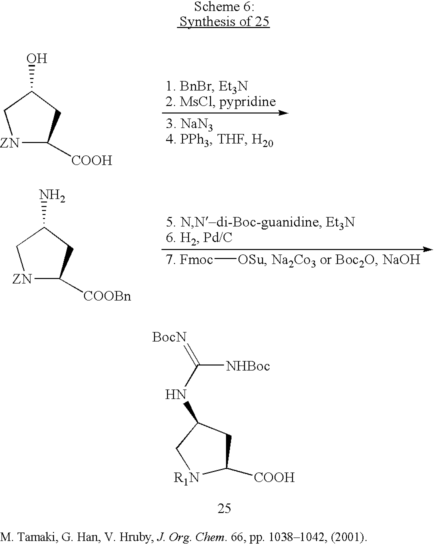

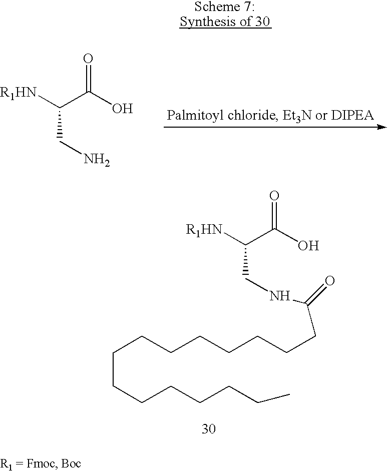

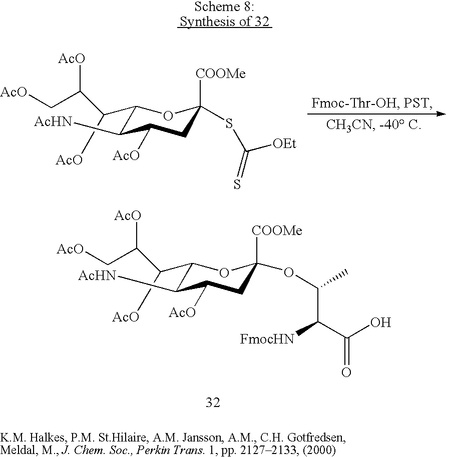

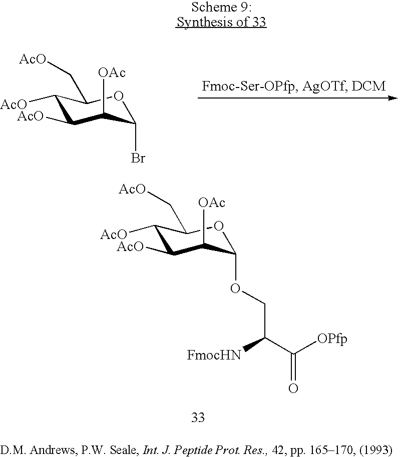

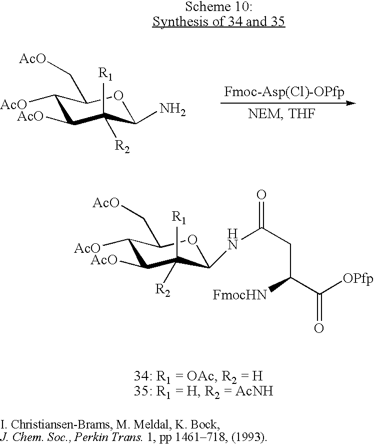

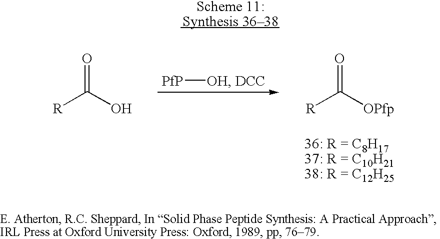

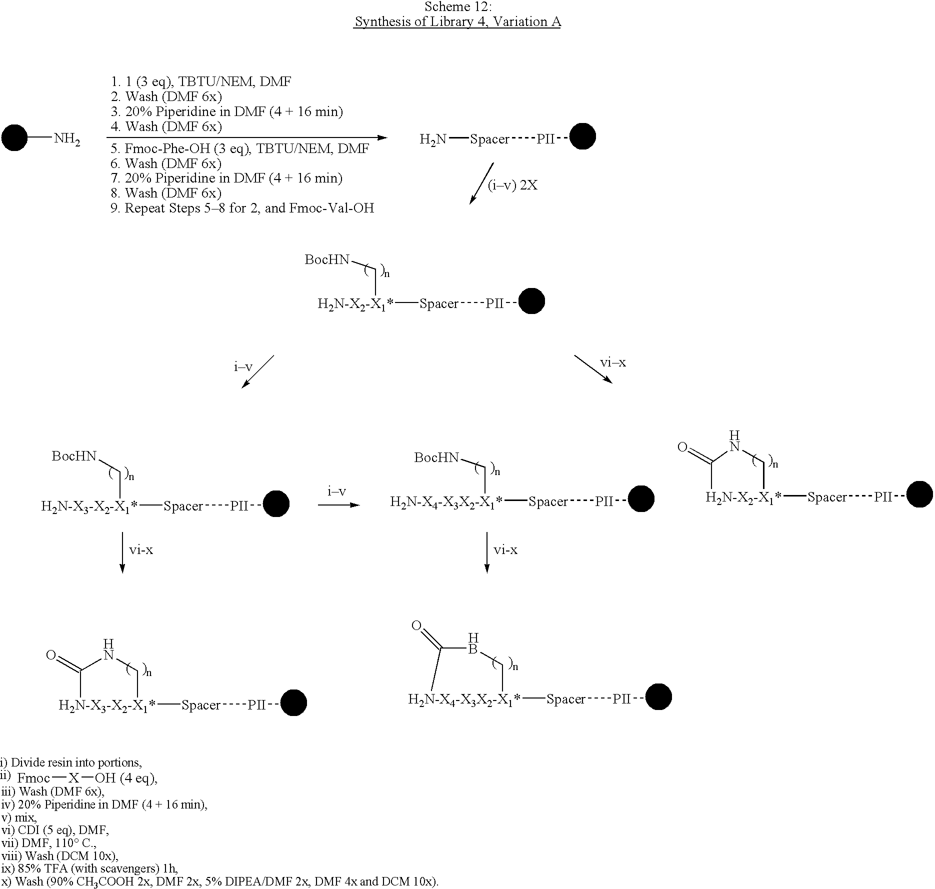

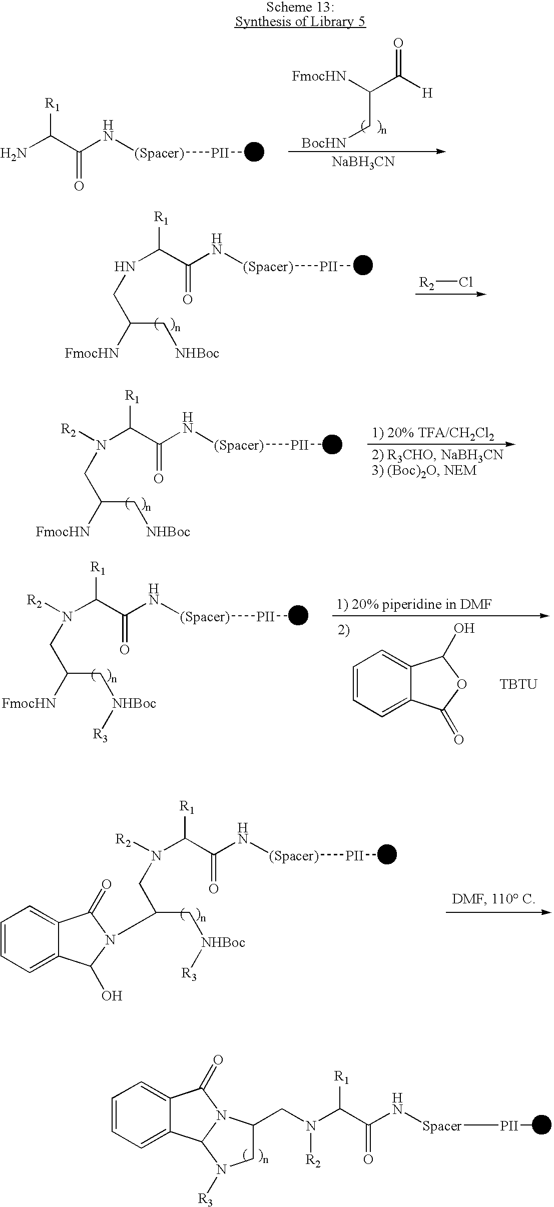

### Example 1

### Synthesis of N-(N′-Fmoc-13-amino-4,7,10-trioxa-tridecyl)-succinamic Acid (2)

N-(N′-Fmoc-13-amino-4,7,10-trioxa-tridecyl)-succinamic acid (2), shown above in Table 1, was prepared as shown in Scheme 5.

4,7,10-Trioxa-1,13-tridecanediamine (5 g, 22.7 mmol, 5 mL) was dissolved in a solution of Na₂CO₃ (7 g) in H₂O (50 mL). Succinic anhydride (2.5 g, 2.5 mmol) in dioxane (50 mL) was added dropwise. The solution turned misty, then into a suspension. It was stirred at room temperature for 24 hours, then heated at 80° C. for another 1 hour. Solvent was removed under vacuum. The residue was treated with 1 N NaOH (200 mL) and extracted with DCM (2×100 mL). The aqueous phase was separated, acidified to pH 1 with 1 N HCl, extracted with DCM (2×100 mL), then neutralized with NaHCO₃ to pH 7.

The crude material was dissolved in 50% acetone/H₂O (120 mL) and Na₂CO₃ (5 g) was added. Fmoc-OSu (7.5 g, 22.3 mmol) was added in portions over 1 hour while pH was kept between 9-10 by addition of 1 M Na₂CO₃. The solution was stirred at room temperature for 18 hours. Acetone was removed under vacuum. The residue was treated with 6 N HCl (60 mL) and extracted with 2×150 1 mL ethyl acetate. The extract was combined and washed with 2×60 mL brine and dried over Na₂SO₄. Solvent was removed under vacuum and the residue was put on a column. Chromatography twice, first with ethyl acetate:hexane (2:1), then DCM/MeOH (3:1) gave pure compound as oil (3.52 g, 29%). The resulting compound (2) showed the following characteristics:

¹H NMR (CDCl₃, δ) 7.76 (d, J=7.2 Hz, 2H), 7.60 (d, J=7.2 Hz, 2H), 7.29-7.43 (m, 4H), 4.40 (m, 2H), 4.23 (m, 1H), 3.46-3.62 (m, 14H), 3.26-3.35 (m, 4H), 2.66 (m, 2H), 2.48 (m, 2H), 1.75 (m, 4H). ¹³C NMR (CDCl₃, δ) 175.1, 172.3, 156.5, 143.7, 140.9, 127.4, 126.8, 124.8, 119.7, 70.0, 69.7, 69.6, 69.2, 68.8, 66.1, 46.9, 38.5, 37.5, 30.5, 29.6, 29.1, 28.5. ES-MS: calcd for C₂₈H₃₈N₂O₈ [M+H]⁺=543.26, found: 543.18.

### Example 2

### Synthesis of (2S, 4S)-N^{α}-Fmoc-4-N,N′-di-Boc-puanidinoproline (25a) and (2S, 4S)-N^{α}-Boc-4-N,N′-di-Boc-puanidinoproline (25b)

(2S, 4S)-N^{α}-Fmoc-4-N,N′-di-Boc-guanidinoproline (25a) and (2S, 4S)-N^{α}-Boc-4-N,N′-di-Boc-guanidinoproline (25b) shown above in Table 2, were prepared from Z-Hyp-OH according to literature procedure described in Tamaki et al., 2001, J. Org. Chem. 66: 1038-1042), as shown in Scheme 6.

### Example 3

### Synthesis of Fmoc-Dapa(Pal)-OH (30a)

Fmoc-Dapa (PAL)-OH (30a) as shown above in Table 2, was prepared as shown in Scheme 7.

Fmoc-Dapa-OH (500 mg, 1.53 mmol) and diisopropylethylamine (780 mg, 6 mmol, 1 mL) were dissolved in DCM (20 mL). Palmitoyl chloride (420 mg, 1.53 mmol, 0.46 mL) was added drop-wise with stirring using a syringe. The suspension slowly became clear. After stirring at room temperature for 2 hours, the solution was concentrated under vacuum. The residue was purified by flash chromatography with DCM:EtOH (10:1) to give pure product (800 mg, 98%) as white powder:

¹H NMR (CDCl₃, δ) 7.68 (m, 2H), 7.49 (m, 2H), 7.19-7.33 (m, 4H), 4.27 (br, 2H), 4.01 (m, 1H), 3.62 (br, 1H), 2.10 (br, 2H), 1.44 (br, 2H), 1.17 (m, 28H), 0.8 (m, 3H). ¹³C NMR (CDCl₃, δ) 176.4, 157.1, 144.1, 143.9, 141.7, 141.6, 128.1, 127.5, 126.2, 125.5, 120.4, 67.7, 47.5, 42.3, 36.7, 32.3, 30.1, 30.0, 29.9, 29.8, 29.6, 23.1, 14.5.

### Example 4

### Synthesis of Boc-Dapa(Pal)-OH (30b)

Boc-Dapa (Pal)-OH (30b) as shown in Table 2, was prepared as shown in Scheme 7.

Boc-Dapa-OH (150 mg, 0.73 mmol) and triethylamine (114 mg, 1 mmol, 0.07 mL) was dissolved in THF (30 mL). Palmitoyl chloride (137 mg, 0.5 mmol, 0.15 mL) was added through a syringe. The solution was stirred at room temperature for 2 hours, then concentrated under vacuum. The residue was purified by flash chromatography with DCM:EtOH (10:1) giving 128 mg (51%) of pure product as white powder:

¹H NMR (CDCl₃, δ) 5.38 (m, 2H), 3.15-3.45 (m, 12H), 2.01-2.10 (m, 4H), 1.18-1.60 (m, 26H). ¹³C NMR (CDCl₃, δ) 173.9, 157.2, 134.4, 79.7, 40.7, 37.9, 36.6, 28.7, 28.6, 26.6, 25.4.

### Example 5

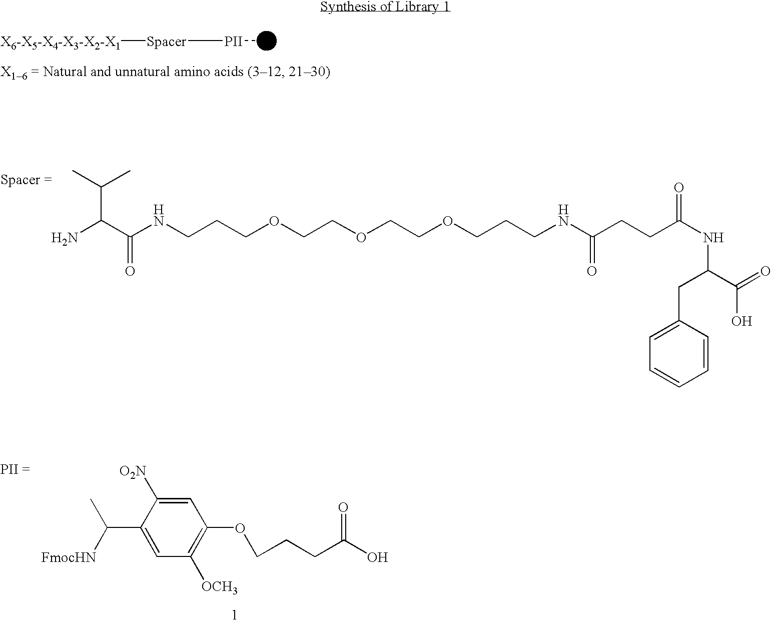

The synthetic scheme for building Library 1 having the structure X₆X₅X₄X₃X₂XI (X=a natural or unnatural amino acid) [SEQ ID NO: 2] is shown above as Scheme 1. Library 1 was prepared on PEGA₄₀₀₀ resin (1 g, 0.12 mmol/g; 300-500 μm beads) using the ladder synthesis method, as previously described in St. Hilaire et al., 1998, J. Am. Chem. Soc. 120: 13312-13320). Since the library was not designed for a particular class of proteins, the building blocks used were chosen arbitrarily but such that as many functional groups as possible were presented in the side chains: e.g. carboxylic acids, amines, indoles, pyridines, aliphatics, aromatics, imidazoles, hydroxyls, and the like. Library 1, X₆X₅X₄X₃X₂X₁, where X=a natural or unnatural amino acid, was produced using building blocks 3-12 and 21-30 [SEQ ID NO: 1]. The building blocks are as shown above in Tables 1 and 2.

To produce Library 1, as shown in Scheme 1, a photolabile linker, PII (1) (3 equivalents) was coupled to the resin beads under TBTU activation. A photolabile linker was chosen because it is stable to a wide variety of conditions and can be readily cleaved to yield a product that does not require further purification before MS analysis. A spacer molecule composed by sequential coupling of Fmoc-Phe-OH, spacer (2) and Fmoc/Boc-Val-OH after TBTU preactivation was then added. The spacer molecule is used to enable the identification of the ligand using MALDI-TOF MS because it increases the mass of the ligand fragments to over 600 mu, i.e. away from the matrix peaks. The spacer was designed to have few or no interactions with any proteins in the mixture.

The six randomized positions of the library were generated using the split and mix approach described in Furka et al., 1991, Int. J. Peptide Protein Res., 37: 487-493 and Lam et al., 1991, Nature, 354: 82-84 in a 20-well custom-made (2.0 mL capacity) multiple column library generator. During the library synthesis, 10% of the growing oligomer was capped using the Boc-protected amino acid analog of the Fmoc Building block. Therefore, a mixture of the Fmoc- and Boc-protected amino acid (90% Fmoc and 10% Boc, 4 equivalents) from stock solutions was activated with TBTU/NEM for 6 minutes and then added to the wells. Coupling times ranged from 4 to 12 hours and reaction completion was determined using the Kaiser test as described in Kaiser et al, 1970, Anal. Biochem., 34: 595-598. After each coupling the resin was pooled, mixed, and divided prior to Fmoc removal. After each coupling and deprotection step, the resin was washed with DMF (10×). After completion of synthesis, the Fmoc group was removed by treatment with 20% piperidine in DMF for 4+16 minutes. The resin was washed with DMF (6×2 minutes), CH₂Cl₂ (10×2 minutes) and then the acid labile side chain protecting groups were removed by treatment with 85% TFA containing 2% triisopropylsilane, 2.5% EDT, 5% thioanisole, 5% water for 1 hour. Then the resin was washed with 90% aqueous acetic acid (4×5 minutes), DMF (2×2 minutes), 5% DIPEA in DMF (2×2 minutes), DMF (4×2 minutes), CH₂Cl₂ (10×2 minutes) and finally methanol (5×2 minutes), before being dried by lyophilization overnight.

### Example 6

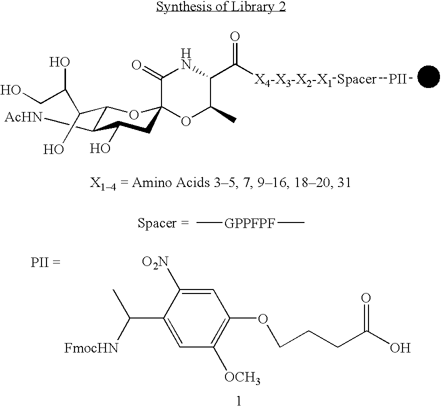

Library 2 containing the peptide X₄ X₃ X₂ X₁ where X is any amino acid of 3-5, 7, 9-16, 18-20, or 31, as shown in Tables 1 and 2 [SEQ ID NO: 3], was synthesized according to Scheme 2, shown above (large black dots represent a resin bead). Library 2 was synthesized on PEGA₁₉₀₀ resin (600 mg, ca. 250.000 beads, 300-500 μm, 0.22 mmol/g loading). The photolabile linker, PII (1) (3 equivalents) under TBTU activation was first coupled to the resin followed by the peptide spacer, GPPFPF [SEQ ID NO: 4], in a syringe, using standard Fmoc-Opfp methodology, for example, as described in Atherton et al., 1989, In: “Solid Phase Peptide Synthesis: A Practical Approach”, IRL Press at Oxford University Press: Oxford, pp. 76-79. The photolabile linker was chosen because it is stable to a wide variety of conditions and can be readily cleaved to yield a product that does not require further purification before MS analysis. The peptide spacer molecule, GPPFPF, is useful to enable the identification of the ligand using MALDI-TOF MS because it increases the mass of the ligand fragments to over 600 mu, i.e. away from the matrix peaks. The spacer was designed to have few or no interactions with carbohydrate binding proteins.

Library 2 was originally designed for binding to carbohydrate binding proteins particularly sialic acid binding proteins, hence the fixed sialic acid threonine lactam in position 5. The building blocks comprising the four randomized positions were chosen from natural amino acids presenting diverse functionalities in the side chain functional group: for example, amides, indoles, aliphatics, aromatics, imidazoles, hydroxyls, and the like. The four randomized positions of Library 2 were generated using the split and mix approach described, for example, in Furka et al., 1991, Int. J. Peptide Protein Res., 37: 487-493) and Lam et al., 1991, Nature, 354: 82-84) in a 20-well custom-made (2.0 mL capacity) multiple column library generator. During the library synthesis, 10% of the growing oligomer was capped using the Boc-protected amino acid analog of the Fmoc Building block. For the coupling of non-glycosylated amino acids, a mixture of the Fmoc- and Boc-protected amino acid (90% Fmoc and 10% Boc, 4 equivalents) from stock solutions was activated with TBTU/NEM for 6 minutes and then added to the wells. Building blocks 3-5, 7, 9-16 and 18-20, as shown in Table 1 above, were used. Coupling times ranged from 4 to 12 hours and reaction completion was checked by the Kaiser test (Kaiser et al, 1970, Anal. Biochem., 34: 595-598).

After each coupling, the resin was pooled, mixed and divided prior to Fmoc removal. After each coupling and deprotection step, the resin was washed with DMF (6×). Building block 32 (2 equivalents), shown in Table 2, was activated with TBTU/NEM for 5 minutes, and then added to all wells overnight. The Fmoc group was removed by treatment with piperidine (4+16 minutes) and the resulting product immediately cyclized to form the lactamized analogue, as evidenced by a negative Kaiser test. The Boc groups were removed by treatment with 10% TFA in DCM for 30 minutes and the carbohydrate acetyl protecting groups were removed by hydrolysis with hydrazine hydrate (55 μL) in methanol (1 ml) for 6 hours, followed by washing with methanol (3×2 minutes), CH₂Cl₂ (3×2 minutes), methanol (3×2 minutes), H₂O (3×2 minutes), toluene (3×2 minutes), and finally diethyl ether (3×2 minutes).

### Example 7

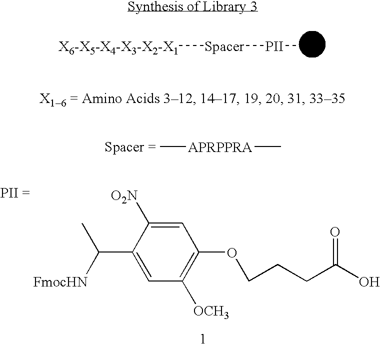

Library 3, a glycopeptide library containing the peptide X₆ X₅X₄ X₃ X₂ X₁ where X is any amino acid of 3-12, 14-17, 19, 20, or 31, (shown in Tables 1 and 2) [SEQ ID NO: 5], was synthesized on PEGA₁₉₀₀ resin (1 g, 300-500 μm beads, 0.23 mmol/g loading) according to Scheme 3. A glycopeptide library was chosen because glycopeptides can mimic oligosaccharides and therefore bind to carbohydrate binding proteins.

The glycopeptides were attached to the resin via photolabile linker, PII (1), and the peptide mass spacer, APRPPRA [SEQ ID NO: 6], was synthesized in a syringe prior to library generation. The photolabile linker was chosen because it is stable to a wide variety of conditions and can be readily cleaved to yield a product that does not require further purification before MS analysis. The peptide spacer molecule, APRPPRA, was used to enable identification of ligand using MALDI-TOF MS, as it increases the mass of the ligand fragments to over 600 mu, away from the matrix peaks, and helps ionization of the fragments because of the arginine content. The spacer was designed to have few or no interactions with carbohydrate binding proteins.

Library 3 was designed for binding to carbohydrate binding proteins, particularly glucose/mannose specific proteins. The building blocks comprising the six randomized positions were chosen from natural amino acids presenting diverse functionalities in the side chain functional group: for example, carboxylic acids, amides, indoles, aliphatics, aromatics, imidazoles, hydroxyls, and the like, as well as glycosyl amino acids bearing mannose and N-acetylglucosamine residues. Natural amino acids were capped with the Boc-protected analog of the Fmoc amino acid while the glycosyl amino acids were capped using aliphatic encoding tags. Amino acids 3-12, 14-17, 19, 20, 31, glycosylated amino acids 33-20, and aliphatic encoding tags 36-38 as shown above in Tables 1 and 2 were used. Randomized positions in the library were generated using the split synthesis approach (Furka, et al., 1991, Int. J. Peptide Protein Res., 37: 487-493 and Lam et al., 1991, Nature, 354: 82-84) using a 20 well custom-made (2.0 mL capacity) multiple column synthesizer.

After each coupling, the resin was pooled, mixed and divided before Fmoc removal with 20% piperidine (2+18 minutes). After each acylation and deprotection step, the resin was washed with DMF (6×). For the coupling of non-glycosylated amino acids, a mixture of the Fmoc- and Boc-protected amino acids (90% Fmoc and 10% Boc, 4 equivalents total) from stock solutions was activated with TBTU/NEM for 5 minutes and then added to the wells. For the coupling of glycosylated amino acids, 3 equivalents of a mixture of the glycosylated amino acid (67%) and the aliphatic encoding tag (33%) was activated with Dhbt-OH and directly added to the wells (33 and 36, 35 and 37, 34 and 38). Coupling times ranged from 4 to 12 hours and reaction completion was checked by the Kaiser test (Kaiser, et al, 1970, Anal. Biochem., 34, pp. 595-598).

After the final coupling, side chain protecting groups were removed using a cocktail consisting of TFA 87.5%, EDT 2.5%, thioanisole 5% and H₂O 5% for 2.5 hours. Then, the resin was washed with 90% aqueous acetic acid (4×5 minutes), DMF (2×2 minutes), 5% DIPEA in DMF (2×2 minutes), DMF (4×2 minutes), CH₂Cl₂ (10×2 minutes), and finally methanol (5×2 minutes), before being dried by lyophilization overnight. Carbohydrate acetyl protecting groups were removed by hydrolysis with hydrazine hydrate (55 μL) in methanol (1 ml) for 6 hours, followed by washing with methanol (3×2 minutes), CH₂Cl₂ (3×2 minutes), methanol (3×2 minutes), H₂O (3×2 minutes), toluene (3×2 minutes), and finally diethyl ether (3×2 minutes).

### Example 8

### Resynthesis of Active Ligands

Ligands for solid phase protein binding were resynthesized on PEGA₄₀₀₀ for the analysis of Myocyte protein and E. coli membrane proteins in the Examples below and on PEGA₆₀₀₀ for the Six-protein mix in the Example below, using standard Fmoc Solid Phase Peptide Synthesis methods as described, for example, in Atherton et al., 1998, In: Solid Phase Peptide Synthesis: A Practical Approach, IRL Press at Oxford University Press: Oxford, pp. 76-79.

### Example 9

### Proliferation of Myocytes

Myocytes were prepared from 1 to 5 day old neonatal Wistar rats (University of Copenhagen) according to literature procedure described in Busk et al., 2002, Cardiovasc. Res., 56: 64-75 and plated into eight P10 culture plates at 6 million cells/plate. Cells were grown at 37° C. and 5% CO₂ humidity in serum free Modified Eagle Media (MEM). After 2 days, the adherent cells were washed at room temperature with serum free MEM (2×) and fresh MEM was added. To four of the plates, 10 μM phenylephrine (PE) was also added. Cells were grown for two more days and then harvested as described below. Cells treated with PE were significantly enlarged at time of harvest.

### Example 10

### Proliferation of Escherichia coli DH 5α-136

A 400 mL culture of Escherichia coli DH 5α-136 (Carlsberg Research Center Collection) was prepared according to the procedures described in Hanahan, 1985, In: DNA Cloning, Vol 1, Glover, D., ed., IRL Press Ltd, pp. 109-135. Cells were grown from innocula in LB media at 37° C. for 5 to 6 hours and harvested in the latter log phase at an optical density of 0.8 (600 nm) by centrifugation (10,000 rpm for 10 minutes at 4° C.). The media containing extracellular protein was retained and the pellet washed once with PBS, pH 7.6 (cellular weight=2.22 g).

### Example 11

### Preparation of Labeled Protein From PE-induced and Basal Myocytes

Protein was extracted from myocytes prepared as described above for Example 9, using a new procedure modified from existing protocols, primarily: Arnott, et al., 1998, Anal. Biochem. 258: 1-18. The media was removed from plates and adhered cells were treated for 10 minutes with ice cold phosphate buffer (0.25 mL, 10 mM, pH 7.5, augmented with 0.15 M NaCl 60 mM Benzamidine HCl, 5 mM EDTA, 10 μg/mL E-64, 10 μg/mL Leupeptin, 10 μg/mL Pepstatin A, and 1 mM PMSF). The cells were scraped off the plates and then lysed (on ice) in a sonicator (2×) using 10 seconds off/10 seconds on cycles.

The resulting suspension was augmented with CHAPS, DTT, and urea to a final concentration of CHAPS (1% w/v), DTT (5 mM), and urea (8 M). After 10-15 minutes on ice, the solution was centrifuged for 10 minutes at 15,000 rpm at 4° C. The supernatant was removed and protein content quantified using the NanoOrange Protein test (Molecular Probes, Eugene, Oreg.): Total protein recovered: 70.5 μg for PE-treated cells and 60 μg for basal cells.

Fluorescent dye Oregon Green 514 (OR) (Molecular Probes) was used to label the healthy/basal cells while Rhodamine Red (RR) (Molecular Probes) was used to label the PE-treated cells. The labeling procedures were carried out according to the manufacturer's protocol. The protein solutions (0.25 mL, 50.4 μg for PE cells and 0.25 mL, 42.8 μg for basal cells) were dialyzed against a solution of 10 mM phosphate buffer, 0.15 M NaCl, pH 7.5 and then 1 M NaHCO₃ (0.025 mL) added to a final pH of 8.5. 10 μL of dye in dry DMF (10 mg/mL) was added and the sample stirred at room temperature for 2 hours. The protein solution was dialyzed extensively against 10 mM phosphate buffer, 0.15 M NaCl, pH 7.5, to remove excess dye.

### Example 12

### Preparation of Labeled Protein From E. coli (Extracellular Protein)

Extracellular protein-containing supernatant obtained from cultured E. coli cells prepared as described above for Example 10 (400 mL broth), was concentrated to 80 mL at 4° C. in an Amicon concentrator using a 6,000 Da molecular weight cutoff (mwco) membrane (Millipore, Bedford, Mass.). The concentrate was further concentrated to 25 mL (protein concentration=94.5 mg/mL) using Amicon microconcentrators centrifuged at 3000 rpm for 1.5 hours. The concentrate was then dialyzed (mwco 10,000 Da) extensively against 10 mM phosphate buffer, pH 6.8 augmented with 0.15 M NaCl, 1 mM ZnCl₂, 1 mM MnCl₂, 1 mM CuCl₂, 1 mM MgSO₄, 1 mM CaCl₂, and 5 mM DTT at 4° C. After the dialysis, a protease inhibitor, PMSF, was added to a final concentration of 1 mM.

The extracellular protein was labeled using an amine reactive dye, succinimidyl N-methylanthranilate (Molecular Probes), using procedures essentially as described above for labeling of myocyte protein in Example 11. 50 mg of the dye in dry DMF (5 mL) was added dropwise with stirring to the extracellular protein solution (25 mL) adjusted to pH 8.35 by the addition of 1 M NaHCO₃ (2.5 mL). The reaction was stirred at room temperature for 2 hours. The reaction was stopped by the addition of 1 M hydroxylamine hydrochloride and stirring continued for another hour. The solution was dialyzed overnight (10,000 Da mwco) at 4° C. against 10 mM phosphate buffer, pH 6.8 containing, 1 mM ZnCl₂; 1 mM MnCl₂; 1 mM CuCl₂; 1 mM MgSO₄; 1 mM CaCl₂.

### Example 13

### Preparation of Labeled Protein from E. coli (Membrane Proteins)

Isolation of E. coli membrane proteins was achieved through modification of published literature procedures: Auer, et al., 2001, Biochemistry, 40:6628-6635, and Molloy, et al., 2000, Eur. J. Biochem. 267:2871-2881. After incubation of E. coli cells for 2 days, washed cells were scraped from plates and centrifuged as described above for Example 10. The cell pellet (approximately 1 g) was suspended in 50 mM Tris HCl, pH 7.5 and pressed (2×) in a French Press at 1500 Psi. The resulting suspension was centrifuged at 2500×g for 10 minutes. The ice cold supernatant was diluted with 2.5 ml of ice-cold 0.1 M sodium carbonate buffer 11 and the solution stirred on ice for 1 hour. Ultracentrifugation was then carried out at 115, 000×g for 1 to 1.5 hours at 4° C., yielding Pellet 1 and Supernatant 1. The membrane pellet 1 was resuspended in 50 mM Tris HCl, pH 7.5 and the pellet was recollected after centrifugation for an additional 20 minutes at 115,000×g, yielding Pellet 2 and supernatant 2. Pellet 2 was solubilized in 50 mM Tris HCl, pH 7.5 containing 10 mM imidazole, 0.5 mM PMSF, 20% glycerol, and 1% Dodecyl Maltoside (DDM) or 33 mM Octyl Glucoside (OG) for 30 minutes at 4° C. The suspension was then centrifuged for 10,000 g for 30 minutes. The pellet and supernatant obtained were labeled Pellet 3 and supernatant 3.

Protein content was determined by checking the absorbance of the protein at 280 nm. The protein concentration in the DDM sample was 0.93 mg/mL, while in the OG sample 0.07 mg/mL protein was obtained. The protein was dialyzed against 10 mM PBS buffer, pH 6.8, containing 1 mM ZnCl₂, 1 mM CaCl₂, 1 mM MnCl₂, and 1 mM MgSO₄, for 1 to 2 hours against three time buffer changes.

The protein was labeled with amino reactive succinylanthranilate dye (blue) DDM (0.49 mg dye) and OG (0.03 mg dye) according to the same protocol used in the extracellular labeling described above for Example 12. The labeling stopping reaction (hydroxylamine hydrochloride addition) was not used in this case, to avoid dilution of the protein. The mixture was dialyzed overnight against 10 mM PBS buffer, pH 6.8 containing 0.01 mM ZnCl₂, 0.01 mM CaCl₂, 0.01 mM MnCl₂, and 0.01 mM MnSO₄, against a three time buffer change.

### Example 14

### Preparation of Labeled Random Mixture of Six Proteins

The following proteins, Concanavalin A (400 μg), Lens culinaris lectin (390 μg), Pisum sativum lectin (300 μg), Wisteria floribunda lectin (260 μg), bovine serum albumin 470 μg, and glyceraldehyde-3-phosphate dehydrogenase, (450 μg), were solubilized in 10 mM PBS augmented with 1 mM CaCl₂, (500 μL) to which was added 1 M Na₂CO₃ (50 μL) for a final pH of 8.3. The protein mixture was labeled with Alexa 488 dye (Molecular Probes) according to the manufacturer's protocol. After stopping the reaction with 1.5 M hydroxylamine (15 μL) the excess dye was removed by washing (6×1 mL) the protein mixture in a centricon YM-10 spun at 5000×g with 10 mM PBS, pH 6.9, augmented with 1 mM CaCl₂ and 1 mM MnCl₂. The protein mixture was washed until the filtrate was no longer fluorescent.

### Example 15

### Solid Phase Screening of Libraries with Labeled Myocyte Proteins

Ligand library 1 (200 mg), prepared as described for Example 5, was transferred to a syringe fitted with a stop-valve and the ligand-beads were washed for 10 minutes (3×) with 10 mM phosphate buffer, pH 6.8, supplemented with 0.15 M NaCl, 1 mM Ca²⁺, 1 mM Zn²⁺, 1 mM Mn²⁺, 1 mM Cu²⁺, and 1 mM Mg²⁺ (3 mL). The ligand-beads were treated with a 1% BSA solution for 30 minutes, then washed with buffer (1×). A mixture of labeled myocyte proteins, including both PE-induced protein (138 μL, 40 μg) and basal protein (167 μL, 40 μg) obtained as described above for Example 11, was prepared in 1.2 mL buffer, and added to the ligand library in the syringe. The proteins and ligand library were incubated at room temperature for 16 hours. The library was then washed with buffer for 5 minutes then with water for 3×5 minutes. The library was examined under a fluorescence microscope and brightly fluorescent red, green, and yellow beads (yellow indicative of both dyes red and green binding; the majority of the beads) were present, as well as unlabelled beads. The fluorescent beads were parted and retained for analysis.

### Example 16

### Solid Phase Screening of Libraries with Labeled E. coli Membrane Proteins

Ligand Library 2 (200 mg) prepared as described above for Example 6, was washed in a 2 ml column (3×10 minutes) with 10 mM PBS buffer, pH 6.8 containing 1 mM ZnCl₂, 1 mM CaCl₂, 1 mM MnCl₂, and 1 mM MgSO₄. To the washed library was added a 1% BSA solution (600 μl), and the BSA incubated with the library for 30 minutes to avoid non-specific binding. The ligand library was then washed, and E. coli labeled membrane protein, prepared as described above for Example 13, was added (0.1 ml). The ligand library and protein mixture was incubated overnight at room temperature. The next day, the beads were washed very well, 5×10 minutes with buffer, and then with water (3×5 minutes). The fluorescence intensity of the beads was analyzed and beads were manually sorted under a fluorescence microscope to obtain 37 fluorescent beads containing protein-ligand binding pairs were obtained for further analysis.

### Example 17

### Solid Phase Screening of Libraries with Labeled Random Mixture of Six Proteins

Ligand Library 3 (150 mg) prepared as described above for Example 7, was washed in a 5 mL syringe (3×10 minutes) with 10 mM PBS buffer, pH 6.8 containing 1 mM CaCl₂ and 1 mM MnCl₂. A solution of 1% BSA (600 mL) was added to the washed library and incubated with the library for 30 minutes to avoid non-specific binding. The six-protein mixture prepared as described for Example 14, in 10 mM PBS buffer, pH 6.8 containing 1 mM CaCl₂ and 1 mM MnCl₂, was then added to the ligand library and incubated for 3 hours and 15 minutes. The beads were then washed very well (5×10 minutes) with buffer and then water (3×5 minutes). The fluorescence intensity of the beads was analyzed and beads were manually sorted in batches under the fluorescence microscope. 112 fluorescent beads containing protein-ligand binding pairs were retained for analysis.

### Example 18

### Sorting of Differentially Fluorescent Labeled Myocyte Protein-Ligand Beads

The fluorescent ligand library beads containing bound myocyte protein obtained as described in Example 15, were sorted using a COPAS (250) NF Bead Sorter (Union Biometrica, Somerville, Mass.). Beads emitting fluorescence of only one color contain proteins from one physical state, proteins expressed only in the PE-induced hypertrophic state (red) or proteins expressed only in the basal state (green). Since the instrument measures one fluorescence emission at a time, beads containing one type of color fluorescence were first sorted and then beads were resorted for emissions from the other fluorescence color. The library was first sorted such that all beads containing significant green fluorescence, basal proteins from healthy myocytes, were isolated. The beads were then resorted to exclude those containing both green and red fluorescence (beads containing proteins from both PE-induced and basal cells). The remaining beads, containing only green fluorescence (proteins from basal cells), were sorted into brightly and less brightly fluorescent beads, to give an indication of either the strength of binding of the ligand to a particular protein or the amount of protein present in the sample binding to the ligand. The beads not containing any green florescence were resorted to isolate those with the highest red fluorescence, for example, beads containing the best ligands binding to proteins expressed only in PE-treated (hypertrophic) cells.

### Example 19

### Identification of Ligands Attached to Fluorescent Beads

After sorting automatically or manually, the labeled protein/ligand beads were washed extensively with 0.1% aqueous TFA to remove residual sheath fluid. The beads were transferred to a stainless steel disc and irradiated with UV light for 1 to 2 hours. Peptide fragments were extracted from the bead with 0.5 μL CH₃CN, then 0.5 μL 70% CH₃CN/H₂O. Another 0.5 μL 70% CH₃CN/H₂O was added to the bead, followed immediately by 0.2 μL MALDI matrix (αc-cyano-4-hydroxycinnamic acid: CHC). The mixture was allowed to evaporate slowly to dryness under a lamp. In most cases, another 0.5 μL 0.1% TFA/H₂O was added to the extract-matrix mixture and dried under a lamp.

The samples thus prepared were used to acquire spectra in the positive reflectron mode of a MALDI time-of flight mass spectrometer (Bruker Reflex III, Bruker-Daltonics, Bremen, Germany). A typical analysis employed 100-300 laser shots. The sequence (hence identity) of the ligand compound on the bead was determined using the instrument's automatic Mass. Diff. program that matches the mass difference between mass peaks with the mass of one of the genetically encoded amino acids. In the case of encoding (such as the use of aliphatic encoding tags) and unnatural amino acid building blocks, the Mass. Diff. program was modified so that the mass difference between mass peaks was also matched with the expected mass difference of the tags and the unnatural amino acids.

### Example 20

### Binding of Differentially Labeled Myocyte Proteins to Identified Ligands

Beads containing 24 different specific ligands identified as specific members of a ligand-protein binding pair in Examples 15, 18, and 19, were placed into 24 wells of a Multiwell filter plate (Multiscreen DV plates, Millipore). Beads containing PEGA₄₀₀₀ resin plus spacer linker with no ligand component were used as a control. Binding was carried out in duplicate with both basal and PE-induced protein, obtained from myocytes and labeled as described above for Example 11. The beads were washed with Millipore water for 3×5 minutes and then with 10 mM PBS buffer (described above for Example 15) for 3×5 minutes under vacuum. PE-induced protein (33 μl) and basal protein (33 μL) were added to each well respectively (10 μg PE and 8 μg Basal protein/well). The plate was covered with aluminum foil and left to incubate at room temperature overnight. The next day the solution containing unbound protein was removed from each well under suction and the beads were washed with Millipore water and 10 mM PBS buffer respectively for 3×5 minutes under vacuum. Fluorescent beads (positive hits) containing ligand-protein binding complexes were observed under a fluorescence microscope and the results documented.

### Example 21

### “Affinity Purification”: Binding of Unlabelled Myocyte Proteins to Identified Ligands

Specific myocyte proteins binding to the identified ligands were isolated by affinity purification. In RNase and DNase free tubes, unlabeled PE-induced protein (32 μL, 13.5 μg) and unlabeled basal protein (49.6 μL, 10.8 μg) were produced, as described in Example 11, and precipitated with acetone (4 volumes) overnight. The pellets were resuspended in Millipore water (PE-130 μl and BASAL-220 μl). Six ligands from PE-induced and Basal positive hits, isolated as described above for Example 20, were chosen and washed as above. To each PE-induced ligand-containing well, a 7 μl unlabeled PE-induced protein solution was added. To each basal ligand-containing well, 8.2 μl of unlabeled basal protein solution was added. The mixtures were incubated overnight at room temperature, and the next day wells were washed with Millipore water to remove unbound protein for 3×5 minutes, then with 10 mM PBS buffer (as described for Example 15) for 3×5 minutes under vacuum.

### Example 22

### Binding of Unlabelled E. coli Membrane Proteins to Identified Ligands

Beads containing 40 different specific ligands that bind E. coli protein, the binding ligands isolated by the process described for Example 16 and identified as described for Example 19, were placed into 40 wells of a Multiwell filter plate (Multiscreen DV plates, Millipore). Control beads containing PEGA₄₀₀₀ resin plus the spacer and linker with no ligand compound were used. The binding of unlabeled E. coli proteins was carried out in duplicate. The ligand beads were washed with Millipore water for 3×5 minutes and then with 10 mM PBS buffer (as described for Example 15) for 3×5 minutes under vacuum. Unlabeled E. coli protein, produced as described for Example 13, (400 μg) was added to each well and incubated at room temperature overnight. The next day, the unbound protein solution from each well was removed under suction and the beads were washed with Millipore water and buffer respectively for 3×5 minutes under vacuum.

### Example 23

### Affinity Purification of Mixture of Six Proteins

Each of eight ligand glycopeptides and peptides attached to PEGA₆000 resin, representative of the positive hits obtained from library screening as described for Example 17, and identified and described by Example 19, were transferred to each of eight 10 mL syringes (4 mL of ligand-resin). The ligand-resin was washed with 10 mM PBS buffer, pH 6.8 containing 1 mM CaCl₂ and 1 mM MnCl₂. A mixture of unlabelled proteins, Glycerol-3-phosphate: BSA: Wisteria floribunda: Lens culinaris: Pisatum sativum in a 4:4:4:1:3:1 weight ratio was dissolved in 10 mM PBS buffer, pH 6.8 containing 1 mM CaCl₂ and 1 mM MnCl₂ The protein mix (3.5 mL, ca. 1.2 mg protein) was applied to each ligand column and allowed to bind overnight. The column was washed with the same buffer until no more protein was eluted (Abs 280 nm). Bound protein was eluted from the column using 0.5M mannose in 10 mM PBS buffer, pH 8.0 containing CaCl₂ and 1 mM MnCl₂ (buffer filtered to remove CaOH₂ formed) for glycopeptides containing only mannose, 0.5 M N-acetylglucosamine in 10 CaOH₂ mM PBS buffer, pH 8.0 containing CaCl₂ and 1 mM MnCl₂ (buffer filtered to remove CaOH₂ formed) for glycopeptides containing only GlcNAc, and both buffers for glycopeptides containing both mannose and GlcNAc, or for unglycosylated peptides. Samples were obtained in about 700 μL volume and frozen for later protein identification.

### Example 24

### Identification of Proteins from Six-Protein Mixture that Bound to Ligand

The identity of each protein eluted from the ligand affinity columns from Example 23 was determined by a combination of Gel electrophoresis and Edman degradation. Gel electrophoresis was carried out using 10% Bis/Tris NuPAGE gels under reducing conditions, using MOPS and then MES buffer. Protein bands were stained using SilverXpress silver staining (NuPAGE). The individual proteins, as well as the mixture, were analyzed along with the eluted fractions. The identity of each protein was obtained by comparison of the band position from the eluted sample to that of each of the known six proteins. The eluted proteins were also identified by N-terminal sequencing of the first 10 amino acids.

### Example 25

### Protein Denaturation Prior to Tryptic Digest

Protein was cleared from beads containing protein-ligand binding “positive hits” isolated as described for Examples 21 (myocyte proteins) and 22 (E. coli proteins). Cleavage was carried out in a similar manner for each bead, using different methods, including enzymatic digests with trypsin, Endoproteinase Arg-C, and endoproteinase Lys-C, and chemical cleavage with CNBr, in order to increase confidence that the correct protein was identified. Cleavage was carried out on several beads or on single beads in tubes, or on single beads resting on a stainless steel disc. In some cases, proteins were denatured and the disulfide bond cleaved prior to tryptic digest, so that the digestion could go to completion. In all cases, similar results were obtained.

A single bead containing a ligand-protein binding complex was treated with 10 M Guanidine HCl (15 μL), 50 mM ammonium bicarbonate buffer, pH 7.8 (3.8 μL), and 20 mM DTT (6.2 μL). The solution was heated at 60° C. for 45-60 minutes. Total reaction volume was 25 μL. After denaturation, the reaction was allowed to cool and 50 mM of ammonium bicarbonate buffer, pH 7.8 (200 μL) was added so that the final concentration of Guanidine HCl was 0.75 M. On-bead tryptic digest was then carried out as described below for Example 26.

### Example 26

### Single Bead Tryptic Digest

A single bead containing ligand and bound denatured or undenatured protein was transferred to an RNase- and DNase-free PCR tube. The bead was washed with 15 μL water for 15 minutes with shaking. Water was removed and the bead was washed with 15 μl 100% acetonitrile on a shaker. The bead was then placed in a speedvac until completely dry. The dry bead was mixed with 15 μL DTT (10 mM in 0.1 M ammonium bicarbonate) at 56° C. for 1 hour. After cooling, the DTT was removed and 50 mM iodoacetamide in 0.1 M ammonium bicarbonate (15 μL) was added. The mixture was incubated in the dark for 30 minutes at room temperature. The iodoacetamide was removed and the bead washed with 30 μL 100% acetonitrile. The bead was dried in the speedvac until dry.

To the dried bead was added trypsin in 50 mM ammonium bicarbonate at a concentration of 12.5 ng/μL. The sample was incubated at 37° C. overnight. The solution was dried and the cleaved peptides were extracted in an Eppendorf tube with 0.4 μL 70% acetonitrile +0.1% TFA, followed by a 0.4 μL acetonitrile and water mixture (2:1)+0.1% TFA, and then 0.5 μL 0.1% TFA. The extracts were combined, and 0.2 μL of the above mixture was transferred to a stainless steel disc to which 0.2 μL of CHC matrix +1.0% TFA was added. In some cases, a range of 0.1-1% of TFA was used as well, as 1% formic acid sometimes facilitates better signals from the sample during MALDI-MS. The remaining solution was transferred to a new tube and stored at −20° C. Alternatively, the bead was placed onto the stainless steel disc after drying and extraction was carried out directly on the stainless steel disc. In both cases, similar results were obtained.

Tryptic digest was also carried out on 3-4 beads in an Eppendorf tube in a similar manner as described for single beads, above in this Example. Similar results were obtained. Proteins were identified as described below for Example 31.

### Example 27

### On-Bead Endoproteinase Asp-N Digest

Proteolytic digestion by Endoproteinase Asp-N was carried out using the protocol described in Sturrock, et al. 1997, Biochem. Biophys. Res. Commun., 236:16-19, with modifications. Typically, after the ligand-bead-bound-protein was subjected to reduction and alkylation performed according to the protocol described in the tryptic on-bead digest method in Example 26, 20 μL of a solution containing 10 μg of endoproteinase Asp-N in 50 mM ammonium bicarbonate buffer, pH 8.0 was added. The reaction was performed for 16 hours at 37° C. The solvents were evaporated and the cleaved peptides were extracted as described above for Example 26, and analyzed as described below for Example 31.

### Example 28

### On-Bead Endoproteinase Lys-C Digest

After the ligand-bead-bound-protein was subjected to reduction and alkylation performed according to the protocol described for the tryptic on-bead digest process for Example 26, a solution of 20 μL containing 10 μg of endoproteinase Lys-C (in 50 mM ammonium bicarbonate buffer, pH 8.0) was added. The reaction was performed for 24 hours at 37° C. The solvents were evaporated and the cleaved peptides were extracted as described for Example 26, and analyzed as described below for Example 31.

### Example 29

### On-Bead Endoproteinase Arg-C Direst

After the ligand-bead-bound-protein was subjected to reduction and alkylation performed according to the protocol described for the tryptic on-bead digest process for Example 26, a solution of 20 μL containing 10 μg of endoproteinase Arg-C (in 50 mM ammonium bicarbonate buffer, pH 8.0) was added. The reaction was performed for 16 hours at 37° C. The solvents were evaporated and the cleaved peptides were extracted as described for Example 26, and analyzed as described below for Example 31.

### Example 30

### On-Bead CNBr Cleavage

CNBr cleavage was performed according to the protocol described in Youngquist et al., 1995, J. Am Chem. Soc., 117:3900-3906. To a single ligand-protein bead in a 500 μL Eppendorf tube was added 15 μL of 20 mg/mL CNBr in 0.1 N HCl. The reaction was allowed to proceed at room temperature in the dark for 14 hours. The samples were dried in a speedvac and cleaved peptides were extracted as described above for Example 26, and analyzed as described below for Example 31.

### Example 31

### Protein Identification

Isolated proteins that bound to specific ligands were identified using peptide mass fingerprinting. Mass spectra were recorded on a Bruker Reflex III MALDI time-of-flight mass spectrometer (Bruker-Daltonics, Bremen, Germany) operated in the positive reflectron mode using delayed extraction. Measurements were performed using the following parameters: Power 83-84 V; lens 7.300. The sum of 200-300 shots was used for each spectrum.

The spectra were calibrated using bradykinin peptide. In some cases, internal mass calibration was performed using a porcine trypsin autolysis product. Peptide masses were searched against peptide mass maps in the National Center for Biotechnology Information (NCBI) database using the following search engines found on the world wide web (www.) for each of:

MS-FIT (prospector.ucsf.edu/ucsfhtml/msfit.htm),

Profound (129.85.19.192/profound_bin/WebProFound.exe), and

MASCOT (matrixscience.com).

A search was performed using the NCBI bacterial (E. coli) database and the mammalian databases for the isolated proteins from the E. coli and myocyte samples, respectively. A molecular mass range was estimated from 0-250 K Da, allowing a mass accuracy that varied from 0.1 Da (some cases 0.3 Da) for each peptide mass. A large pI range from 0-14 or 0-12 was considered for each search. If no proteins matched, the mass window was extended. Partial enzyme cleavages allowing for two missed cleavage sites and modification of cysteine by alkylation were considered in the search approaches. A protein was considered identified if the matched peptides covered at least 30% of the complete sequence. A match of less than 30% was considered in some cases, if prominent peaks were obtained. Usually, four or more peptides were used for identification. In some instances, hypothetical proteins or gene products, such as biochemical material, either RNA or protein, calculated from the expected expression of a gene and to which a function may be assigned based on sequence homology, were identified.

### Example 32

### Results for Myocytes/Ligand Library 1

Using the procedures described above for Examples 1-5, the ligand Library 1 (containing unnatural amino acids) as described for Example 5, myocyte proteins prepared and labeled as described for Examples 9 and 11, screening as described for Example 15, sorting and identifying as described for Examples 18 to 21, digestion and protein identification as described for Examples 25-31, previously unknown, specific, differential ligand-protein binding pairs were identified for the normal (basal) myocyte protein mixtures and the phenylephrine (PE)-treated myocyte proteins screened against the ligands of Library 1. Phenylephrine was used to provide an in vitro model of hypertrophy, for example, cardiac hypertrophy (Arnott et al., 1998, Anal. Biochem., 1:1-18).

The results shown in Table 4 below demonstrate that the process of the invention can be successfully used to identify membrane proteins such as ion channels, symporters, and G-protein coupled receptors, together with specific ligands that bind to them, in one quick step. This is an important result in light of that fact that at least 50% of all drug targets are membrane proteins. Low abundance proteins, i.e., proteins with a codon bias of <0.1, such as transcription factors, protein kinases, and phosphatases (See, for example, Gygi et al, 2000, Curr. Opin. Biotechnol., 11 pp. 396-401), were also detected. The observation that more than one protein binds to a ligand implies that either each of the proteins identified bind to the same ligand or, in some cases, proteins that work together and interact with each other (i.e. protein complexes; e.g. Entries 1 and 5) were isolated. The type of library used for screening restricts the number and type of proteins that can be identified. In practice, several different types of libraries are screened with the same protein mixture. The described procedures for library synthesis, screening, sorting, and identifying both ligand and protein can be readily automated using known procedures to render these procedures truly “high-throughput”.

The proteins listed in Table 4 represent some of the proteins that are more abundant in one cellular state than the other. In this example, Entries 1-6 are proteins that are primarily present in basal cells but not in hypertrophied cells, while Entries 7-12 show the reverse situation. Both sets of proteins are therefore important in the etiology of cardiac hypertrophy and identify these specific ligand:protein pairs for use in development of new therapeutics for disease, for example, cardiac hypertrophy.

The ligands identified also provide an important tool for furthering an understanding of hypertrophy disease at the molecular level. Some of the proteins identified as binding these ligands are useful as biomarkers for cardiac hypertrophy and related disease, aiding diagnosis. The traditional therapeutic modality for the amelioration of cardiac disease including hypertrophy is primarily through the use of angiotensin converting enzyme (ACE) inhibitors, β-blockers, and ion channel modulators. In Table 4, Entries 5 and 11 are ion channels identified as binding proteins, and the effect of these on cardiac hypertrophy can now be investigated using the identified ligands. Furthermore, since ion channels are important in a wide range of diseases (e.g. epilepsy and hypertension) the identified ligands provide design templates for new drug candidates for existing diseases related to identified ion channels. It is known, for example, that one of the proteins identified by this process (see table below), myosin light chain kinase, is important in the etiology of cardiac hypertrophy (Aoki et al, 2000, Nat. Med., 6: 183-188). From genomic analysis of the genes affected in alternative models of cardiac hypertrophy, the genes that were enriched in load-induced hypertrophy and in neonatal hearts (hypertrophic state) included genes coding for protein phosphatase 1 gamma, mitochondrial NADH-dehydrogenase, and the 60S ribosomal protein L3 (Johnatty et al, 2000, J. Mol. Cell. Cardiol., 32: 805-825). In addition, mitochondrial ATP synthase gene expression in mice was down regulated after induction of hypertrophy with isoproterenol (Friddle et al, 2000, Proc. Nat. Acad. Sci., 97, 6745-6750). These proteins or closely related ones were identified as specific binding proteins in this Example, together with specific binding ligands (see Table 4, Entries 4, 7 and 11). Proteins of unknown identity or function in cardiac hypertropyhy were also observed (e.g. SPTR proteins, G-protein receptor used for signaling hypothetical proteins). The partnering ligands identified for these proteins can be used to elucidate their importance in cardiac hypertrophy.

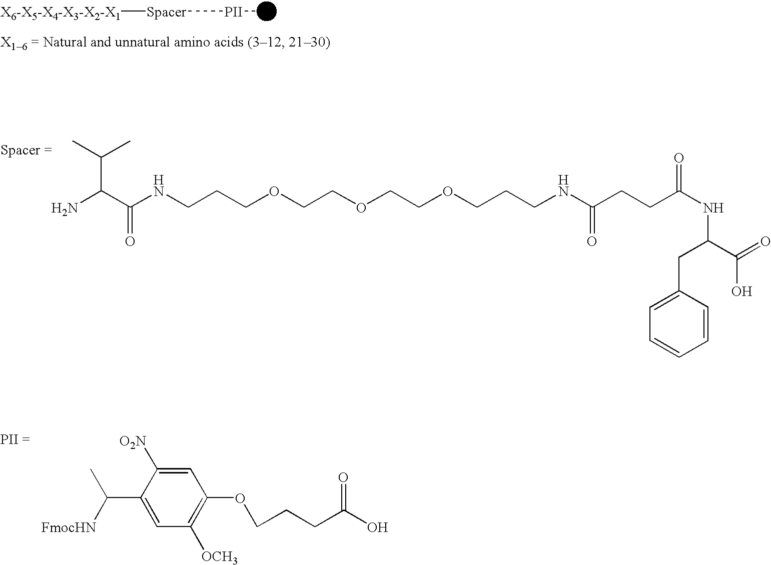

TABLE 4
List of identified ligands and proteins for Library 1 and myocyte proteins.

| | | |
|---|---|---|
| Entry | Identified Ligand | Identified Protein(s) |

Normal Myocytes

| | | |
|---|---|---|
| 1 | Pip-Pal-Pal-Phe-Pya-Pip | Myosin light chain kinase (LA, MA); (Tyrosine phosphatase (LA, M); |
| | [SEQ ID NO: 7] | ATP Synthase component (M)) Gluthathione S-transferase; Cytochrome |
| | | P450 (M); Ribosomal proteins (60s); SPTR (M)) |
| 2 | Pya-Hyp-Hyp-Phe-Acm-Tyr | Troponin T; Growth hormone receptor (M); Protein kinase (LA) |
| | [SEQ ID NO: 8] | |
| 3 | Pya-Gua-Pip-Acc-Phe-Pip | NADH dehydrogenase; ATP binding component (M); Myosin; Histone |
| | [SEQ ID NO: 9] | associated proteins (M) |
| 4 | Phe-Aze-Gly-His-Gly-Aze | Hypothetical proteins; Cysteine and tyrosine rich proteins of unknown |
| | [SEQ ID NO: 10] | function (M); (Mitochondrial ATP synthase; Ribosomal proteins (L |
| | | series);) Serine protease; SPTR. |
| 5 | Phe-Thr-Pya-Pip-Asp-His | (Sodium channel (M); Chloride channel (M)) Troponin; Ribosomal |
| | [SEQ ID NO: 11] | proteins L26; Serine hydroxy methyl transferase; Zn Finger protein (MA); |
| | | Adherin aminotransferase (M); (Gluthathione transferase; Gluthathione |
| | | peroxidase;). |
| 6 | Phe-Ppy-Acc-Ala-Ppy-Hpy | Hypothetical proteins; Troponin T; (Phospholipase C (MA); |
| | [SEQ ID NO: 12] | Phosphatidylcholine sterol acyl transferase). |

Phenylephrine Treated (Hyperthrophic) Myocytes

| | | |
|---|---|---|
| 7 | Phe-Abi-Pal-Hyp-Thr-Hyp | Zinc finger associated protein; Ribosomal proteins; Protein phosphatase |
| | [SEQ ID NO: 13] | (LA) |
| 8 | Phe-Gua-Pal-Tyr-Gua-Tyr | Glucose-6-Phosphatase; Succinate dehydrogenase; ARL-interacting |
| | [SEQ ID NO: 14] | protein (M); SPTR (M), Nucleic acid binding protein. |
| 9 | Pal-Abi-Gly-Gly-Abi-His | Ribosomal protein (60s + 40s); Low density lipoprotein receptor |
| | [SEQIDNO: 15] | |
| 10 | Abi-Thr-Hyp-Hyp-His-?- | Phosphofructokinase; Selenium binding protein; (Serine arginine rich |
| | [SEQ ID NO: 16] | protein kinase (LA); Guanylate kinase (LA), Protein tyrosine kinase |
| | | (LA)); Alkaline phosphatase; Symporter (M); SPTR (M); WAP-protein; |
| | | GTP Hydrolase, Actin filament. |
| 11 | Pya-Gua-Abi-Asp-Abi-Tyr | SPTR (M); Ribosomal proteins (60s); (Calcium channel (M); Slo channel |
| | [SEQ ID NO: 17] | protein isoform (M); Potassium conductance calcium activated channel |
| | | (M); Symporter (M;) NADH dehydrogenase; Malate dehydrogenase; N- |
| | | Acetyl transferase; Mitochondrial associated protein; G-protein receptor |
| | | used for signaling (LA, M). |
| 12 | Abi-Phe-Abi-Phe-Che-Tyr | Cathepsin E; Ribosomal proteins, Actin binding protein, Amino acid |
| | [SEQ ID NO: 18] | transferase. |

In the table above,
Abi = 3-amino-3-(biphenyl)-propanoic acid;
Acc = 3-Amino-carboxymethyl-caprolactame;
Aze = L-Azetine-2-carboxylic acid;
ARL = ADP-ribosylation like factor;
Che = 1-amino-1-cyclohexanecarboxylic acid;
Gua = 4-S-(Di-Boc-Guanidino)-L-Proline;
Hyp = L-Hydroxyproline-OH;
Pal = L-Dapa(Palmitoyl)-OH;
Pip = 4-Phenyl-Piperidine-4-carboxylic acid;
Ppy = 5-Phenyl-Pyrrolidine-2-carboxylic acid;
Pya = L-3-PyridylAla-OH;
SPTR = Hypothetical membrane transporter proteins;
WAP = Whey acidic protein.
Possible protein complexes, protein families of proteins that work closely together are enclosed in parentheses. The large filled circle represents a bead of resin. The letters in parentheses have the following designation: LA: low abundance proteins, M: protein is a transmembrane one or partially embedded. MA: protein is associated with a membrane.

### Example 33

### Results for E. coli Protein/Ligand Library 2

Using the processes and procedures described for the Examples above, the sialic acid lactam Library 2 of Example 6, and E. coli membrane proteins (i.e. protein extract from E. coli that has been processed to access primarily inner and outer membrane proteins) produced, isolated and labeled as described in Examples 10 and 13 were mixed together and specific ligand protein binding pairs attached to resin beads were isolated as described in Example 16. The identity of the ligands were established by MS as described in Example 19, ligands were resynthesised on solid phase as in Example 8 and the protein binding partners isolated on the resin bead as detailed in Example 22. The identity of the protein binding partners for each ligand was determined by first denaturing the protein bound to the bead as in Example 25, followed by tryptic digest of one or several beads as described in Example 26 and the resulting peptides used to search databases for the identity of the proteins as described in Example 31. Of the 37 ligands isolated, 34 were conclusively identified and used for the isolation and identification of the bound proteins. The specific pairs identified thus far are shown in Table 5 below, where T(Sa) Sialic acid threonine lactam (see Table 2 for specific ligand structures), The letters in parentheses have the following designation: LA: low abundance proteins, M: protein is located either on the inner or outer membrane and can be transmembrane or partially embedded, P: proteins primarily located in the periplasmic space.

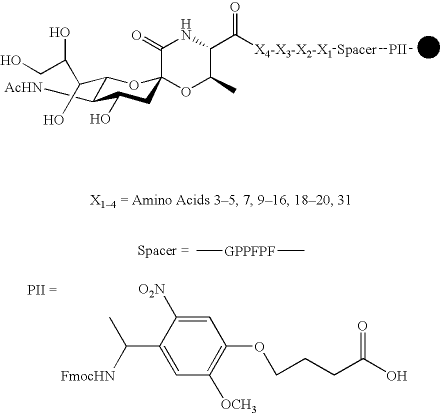

TABLE 5
List of identified ligands and proteins for
Library 2 and E. coli membrane proteins.

| | Identified | |
|---|---|---|
| Entry | Ligand | Identified Protein(s) |
| 9 | T(Sa)-F-N-H-S | Phosphate acetyl transferase, acid shock protein, molybdopterin converting factor |
| | [SEQ ID NO: 19] | subunit |
| 10 | T(Sa)-F-A-L-V | Chaperone DnaK, putative hydrolase, transposase |
| | [SEQ ID NO: 20] | |
| 11 | T(Sa)-F-G-I-W | Histidine synthetase, aspartate carbamoyl transferase, protein involved in permease |
| | [SEQ ID NO: 21] | transport system (M) |
| 12 | T(Sa)-F-G-I-M | Transposase, transcriptional regulator (LA), GroEL, protein involved in the taurine |
| | [SEQ ID NO: 22] | transport system (M) |
| 13 | T(Sa)-G-V-F-L | 50 S ribosomal protein, heme binding lipoprotein (M), regulator for D-glucarate, D- |
| | [SEQ ID NO: 23] | glycerate and D-galactarate, glutamine tRNA synthetase |
| 14 | T(Sa)-Y-S-M-P | Biotin synthetase, UDP-glucose dehydrogenase, tyrosine protein kinase (LA), fatty |
| | [SEQ ID NO: 24] | acid oxidase complex proteins |
| 15 | T(Sa)-L-S-W-W | NAD-dependent 7-alpha-hydroxysteriod dehydrogenase, homocysteine transferase, |
| | [SEQ ID NO: 25] | nitrate reductase (P), lactate dehydrogenase, citrate synthetase |
| 16 | T(Sa)-H-W-H-I | Mannose-1-phosphate guanyl transferase, isopropyl malate dehydrogenase (M) |
| | [SEQ ID NO: 261 | |
| 17 | T(Sa)-H-W-V-V | Pyruvoyl dependent aspartate decarboxylase, colicin E2, Histidine kinase (M) |
| | [SEQ ID NO: 27] | |
| 18 | T(Sa)-H-L-G-Y | Protein involved in lipopolysaccharide biosynthesis (M), phosphomannose isomerase |
| | [SEQ ID NO: 28] | |
| 19 | T(Sa)-I-Y-L-F | Membrane bound ATP synthetase (M), ATP hydrolase |
| | [SEQ ID NO: 29] | |
| 20 | T(Sa)-F-G-L-M | Hemolysin C, high affinity potassium transport system (M), quinone oxidoreductase |
| | [SEQ ID NO: 30] | (M), ferrodoxin dependent NA(D)PH oxidoreductase (M) |
| 21 | T(Sa)-W-V-N-M | Transposase, inner membrane protein for phage attachment (M) |
| | [SEQ ID NO: 31] | |
| 22 | T(Sa)-M-V-N-W | ATP dependent helicase, mob C |
| | [SEQ ID NO: 32] | |
| 23 | T(Sa)-H-I-G-Y | Fimbrial subunit, outer membrane pyruvate kinase (LA, M) |
| | [SEQ ID NO: 33] | |
| 24 | T(Sa)-L-Y-L-F | Fimbrial protein precursor, alkaline phosphatase (P), cytochrome related proteins (P), |
| | [SEQ ID NO: 34] | |
| 25 | T(Sa)-H-W-H-L | Chorismate mutase, xanthine dehydrogenase, carbamoyl phosphate synthetase |
| | [SEQ ID NO: 35] | |
| 26 | T(Sa)-F-V-W-H | NADH dehydrogenase (M), protein involved in flagellar biosynthesis and motor |
| | [SEQ ID NO: 36] | switching component (M) |
| 27 | T(Sa)-F-V-W-H | Lysine-arginine-ornithine-binding protein (P), ATP-binding component of glycine- |
| | | betaine-proline transport protein (P) |
| 28 | T(Sa)-L-Y-I-F | Colicin, hypothetical membrane protein (M) outer membrane lipoprotein (M), |
| | [SEQ ID NO: 37] | arylsulfatase |

The results of Example 33 shown in Table 5 demonstrate that the process of the invention can be successfully used to identify membrane proteins and specific binding ligands in one quick step. This is an important result in light of that fact that at least 50% of all drug targets are membrane proteins. The proteins identified were inner and outer membrane proteins as well as proteins from the periplasmic space and a few from the cytosol.

Proteins with a wide range of functions, including transport (e.g. protein involved in taurine transport system), protein synthesis (transposase and Chaperone DnaK), metabolism (chorismate mutase, citrate synthetase), and lipopolysaccharide biosynthesis (protein involved in lipopolysaccharide biosynthesis) were identified as well as proteins of as yet unknown function. The type of library used restricts the number and type of proteins that can be identified. In practice, several different types of libraries are screened with the same protein mixture. For more complex cell types, where, for example, thousands of proteins will be isolated, the described processes can be readily automated, using known methods.

Of the proteins identified from this analysis, one is a proven target for antibacterial drugs. The 50 S ribosomal protein (Table 5, Entry 13) is a target of chloramphenicol and macrolide antibiotics that block bacterial protein synthesis (See, for example, Section 13: Infectious disorders, Chapter 153: Antibacterial drugs, In: The Merck Manual of Diagnosis and Therapy, M. H. Beers and R. Markow (Eds), 17^{th} ed. 1999, Merck & Co). In light of increasing bacterial resistance, there is an urgent need to develop alternate antimicrobials. Current approved antibiotics target 15 bacterial enzymes and macromolecular complexes (Strohl, W. R. (Ed): Biotechnology of antibiotics. Marcel Dekker, New York, 1997) in the area of cell-wall biosynthesis, cell membrane permeability, protein synthesis and DNA replication and repair synthesis (Section 13: Infectious Disorders, Chapter 153: “Antibacterial drugs,” M. H. Beers and R. Markow (Eds), 17^{th} ed. 1999, Merck & Co.). In the present invention, all of the proteins identified in this Example are putative drug targets for antibacterials. This premise can be rapidly tested in biological assays using the matching ligand-binding partner that has also been identified. Thus, all the binding ligands identified are putative antibacterial agents, providing that they selectively interact with bacterial proteins in the host or interact with a bacterial protein for which there is no human counterpart.

In the age of genomics, where the complete genomes of several pathogens are known, several putative, novel antibacterial targets have been postulated after intensive sequence analysis. Some of these newly recognized antibacterial drug targets have also been identified in this Example and are mentioned below. Histidine kinase (Table 5, Entry 17) has recently been recognized as a target protein for antimicrobial agents. (Matsushita et al., 2002, Bioorg. Med. Chem., 10: 855-67; Deschenes et al., 1999, Antimicrob. Agents Chemother., 43: 1700-03; Lyon et al., 2000, Proc. Nat. Acad. Sci., 97: 1330-35). A Philadelphia-based company, Chaperone Technologies (see the world wide web site: chaperonetechnologies.com/technology.htm), is based on the development of antimicrobial drugs from compounds that bind to DnaK chaperone (Table 5, Entry 10). The pharmaceutical company former SmithKline Beecham has developed methods of using nitrate reductase alpha subunit (Table 5, Entry 15) to screen for antibacterials effective against S. aureus (Molecular Targets, 2001, 12, pp. 13; web site: experts.co.uk). Phosphomannose isomerase (Table 5, Entry 18) is an essential enzyme in the synthesis of GDP-mannose that is utilized in the synthesis of lipopolysaccharides, glycoproteins, and exopolysaccharides (Wills et al, 2000, Emerging Therapeutic Targets, 4(3): 1-30). This enzyme has been recognized as a potential drug target for antifungals and in Candida, has been inhibited by sulfadiazene (Wells et al, 1996, Biochemistry, 34, pp. 7896-7903). All the aminoacyl tRNA synthetases are putative targets for antibacterial agents. In fact, the approved antibiotic, Mupirocin, inhibits the enzyme isoleucine tRNA synthetase, (Section 13: Infectious disorders, Chapter 153: Antibacterial drugs, In The Merck Manual of Diagnosis and Therapy, M. H. Beers and R. Markow (Eds), 17^{th} ed. 1999, Merck & Co.) for which the glutamine analog has been identified by this Example of the invention (Table 5, Entry 13).

From microbial genomic analysis, several classes of proteins, such as outer membrane proteins, host-interaction factors, permeases, metabolic enzymes, DNA replication and transcription and repair apparatus, have been identified as putative antibacterial drug targets (M. Y. Galperin and E. V. Koonin, 1999, Curr. Opin. Biotechnol., 10: 571-578) and are among those binding proteins detected by this Example of the invention and are mentioned below. An analysis of virulence factors, potential drug targets in H. pylori and N. meningitidis, showed that carbamoyl phosphate synthetase (Table 5, Entry 25), NADH-ubiquinone oxidoreductase (cf. Table 5, Entry 20), fimbrial protein (Table 5, Entries 23 and 24), LPS biosynthesis protein (Table 5, Entry 18), and ATP dependent helicase (Table 5, Entry 22) were promising targets (Junaid Gamieldien, Ph.D Dissertation , Chapter 4: “Novel Approaches for the Identification of Virulence Genes and Drug Targets in Pathogenic Bacteria”, 2001, University of Western Cape, South Africa). Furthermore, knockout analysis of selected genes in H. pylori showed that knocking out the gene for carbamoyl phosphate synthetase resulted in decreased colonization efficiency while the gene coding for NADH-ubiquinone oxidoreductase proved essential for the survival of the bacteria. Finally, enzymes in the aromatic amino acid biosynthetic pathway that are absent in humans make attractive drug targets. One such enzyme, chorismate mutase (Table 5, Entry 25) has been identified by this Example of the invention. Other proteins identified by this process have diverse functions that may be essential to the survival of the bacteria, or as yet unknown functions. The function of these proteins can be probed using the identified ligands as a starting point.

### Example 34

### Results for Six Protein Mixture and Ligand Library 3

Using the processes and procedures described for the Examples above, the glycopeptide Library 3 of Example 7, and the six protein mixture: Con A, P. sativum lectin, L. culinaris lectin, W. floribunda lectin, Glyceraldehyde 6-phosphate, and bovine serum albumin (BSA) labeled as described in Example 14 were mixed together and specific ligand protein binding pairs attached to resin beads were isolated as described in Example 17. The identity of the ligands were established by MS as described in Example 19, ligands were resynthesised on solid phase as in Example 8 and the protein binding partners isolated on the resin bead as detailed in Example 23. The identity of the protein binding partners for each ligand was determined by a combination of gel electrophoresis and Edman degradation as described in Example 24.

The identified ligand-protein pairs are shown in the Table 6 below, where ManS=Mannose linked to hydroxyl group of Ser; ManN=Mannose amine linked to the side chain carboxyl group of Asp (N^{α}-Mannosylasparagine) and GlcNN=N-Acetylglucosamine linked to the side chain carboxyl group of Asp (N^{α}-N-Acetylglucosaminylasparagine) (see Table 2 for exact structures). The large filled circle represents a resin bead.

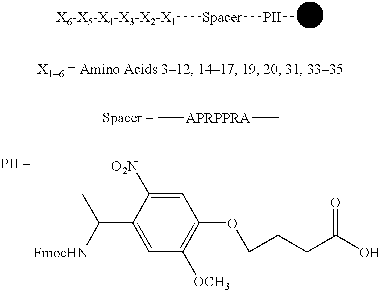

TABLE 6
List of identified ligands and proteins
for Library 3 and six protein mixture.

| Entry | Identified Ligand | Identified Protein(s) |
|---|---|---|
| 1 | ManS-Gly-ManS-Asp-Asn-Ala | Con A, P. sativum lectin |
| | [SEQ ID NO: 38] | |
| 2 | ManS-GIy-GlcNN-Asn-ManS-Tyr | Con A, P. sativum lectin, |
| | [SEQ ID NO: 39] | L. culinaris lectin |
| 3 | ManN-Phe-Trp-Ser-Lys-His | Con A, P. sativum lectin |
| | [SEQ ID NO: 40] | |
| 4 | GlcNN-Trp-Phe-Asp-Trp-Pro | Con A |
| | [SEQ ID NO: 41] | |
| 5 | GlcNN-Val-GlcNN-His-ManS-Gly | Con A, P. sativum lectin |
| | [SEQ ID NO: 42] | |
| 6 | ManN-ManS-ManN-Trp-Ser-Trp | Con A, P. sativum lectin, |
| | [SEQ ID NO: 43] | L. culinaris lectin |
| 7 | Gly-Pro-Lys-Lys-Tyr-His | Con A, P. sativum lectin, |
| | [SEQ ID NO: 44] | L. culinaris lectin |
| 8 | His-Thr-Trp-Gly-Tyr-Trp | Con A |
| | [SEQ ID NO: 45] | |

In this Example, some of the ligands and matching binding proteins identified are useful glyco-tool for elucidating the molecular mechanisms of lectin-ligand binding and molecular mimicry. It is interesting that GicNN-Trp-Phe-Asp-Trp-Pro binds only to Con A and not to the other lectins, although they are reported to have similar specificity for a sugar-containing ligand. In addition, ligand 8, which does not contain a sugar residue, binds selectively to Con A, providing a tool for the study of molecular mimicry in lectin-ligand interactions. All the ligands identified, when attached to chromatographic resins (e.g. sephacryl, sepharose), are useful for affinity purification of the three lectins used in the study (some selectively). These identified binding ligands are also useful to purify novel mannose/glucose specific lectins that may be used for large-scale commercial production of proteins that bind specifically to lectins, including antibodies for clinical use and other glycoproteins.

### Example 35

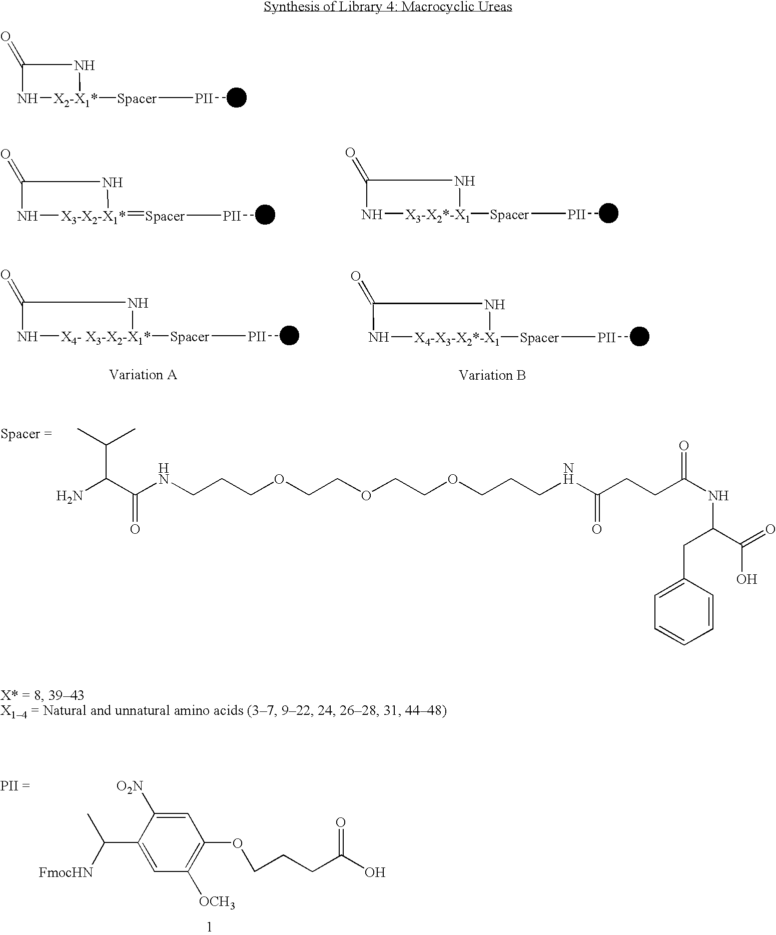

In one embodiment, Library 4, shown above, is prepared on PEGA₄₀₀₀ resin (2 g, 0.1 mmol/g; 500-700 μm beads) using the ladder synthesis method, as previously described in St. Hilaire et al., 1998, J. Am. Chem. Soc. 120: 13312-13320. In alternative embodiment, Library 4 can be synthesized without the ladder, for example, omitting the spacer. The synthesis of variation A is shown in Scheme 12, where for peptides X₂ X₁ [SEQ ID NO: 46], X₃ X₂ X₁ [SEQ ID NO: 47], and X₄ X₃ X₂ X₁ [SEQ ID NO: 48], X₁ is chosen from amino acids 8, 39-43, and X₄ X₃ X₂ are each independently chosen from amino acids 3-7,9-22, 24, 26-28, 31, and 44-48, as shown in Tables 1 and 2. The synthesis of variation B is carried out similarly, where for peptides X₃ X₂ X₁ [SEQ ID NO: 49], and X₄ X₃ X₂ X₁ [SEQ ID NO: 50], X₂ is chosen from amino acids 8, 39-43, and each of X₁, X₃, X₄ is chosen from amino acids 3-7,9-22, 24, 26-28, 31, and 44-48, as shown in Tables 1 and 2.

It has been shown that cyclic and aliphatic urea containing compounds are inhibitors of Cdk4 kinase (see, for example, Dolle, 2002, J. Comb. Chem., 4: 369-418). It is therefore expected that a library of peptidic cyclic ureas such as Library 4 binds primarily to kinases present in the cellular protein mixture used for screening. Since no particular kinase is targeted, the library is not designed based on structure-activity function data. The building blocks used are chosen arbitrarily, and in a manner to present as many functional groups as possible in the side chains: including, for example, carboxylic acids, amines, indoles, pyridines, aliphatics, aromatics, imidazoles, hydroxyls. It is expected that proteins that are not kinases will also bind to some of the Library members. The building blocks used, 3-7,9-22, 24, 26-28, 31, and 44-48, are shown in the Tables 1-3 above.

The photolabile linker, 1 (3 equivalents) is coupled under TBTU activation. A spacer molecule formed by sequential coupling of Fmoc-Phe-OH, compound 2, and Fmoc/Boc-Val-OH after TBTU preactivation, is then added to the linker. The spacer molecule is used to enable the identification of a ligand using MALDI-TOF MS, as the spacer increases the mass of the ligand fragments to over 600 mu, away from the matrix peaks. The spacer is designed to have few or no interactions with proteins in the mixture. Where no spacer is used, the first set of randomized amino acids is coupled directly to the photolabile linker. The library compounds with no spacer and ladder fragments are analyzed using tandem Mass spectrometry and/or magic-angle-spinning (MAS) NMR.

Randomized positions of the library are generated using the split and mix approach described in Furka et al., 1991, Int. J. Peptide Protein Res., 37: 487-493 and Lam et al., 1991, Nature, 354: 82-84 in one or more 20-well custom-made (2.0 mL capacity) multiple column library generator. In the ladder synthesis strategy, 5% of the growing oligomer is capped using the Boc-protected amino acid analog of the Fmoc building block. Therefore, a mixture of the Fmoc- and Boc-protected amino acid (95% Fmoc and 5% Boc, 4 equivalents) from stock solutions are activated with TBTU/NEM for 6 minutes and then added to the wells. In the case of no ladder synthesis, only Fmoc protected building blocks (4 equivalents) are used.

Library 4 contains variations in the position and size of the cyclic urea formed and the positional variation is designated A and B. In variation A, in the first position, six different amines (8, 39-43) are coupled to the spacer or linker. After mixing and deprotection of the Fmoc protecting group by treatment with 20% piperidine in DMF for 4+16 minutes, 20 different building blocks are coupled. One third of the resin is then removed and the Fmoc protecting group removed. The N-terminal amine is then treated with carbonyldiimidazole (CDI) (5 equivalents) in DMF for 1.5 hours at room temperature. The resin-bound product is then heated to 110° C. in DMF for 2 hours to promote cleavage of the Boc protecting group and simultaneous cyclization to form the urea. After a resin mixing step, the Fmoc group of the remaining two-thirds of the resin is cleaved and 20 building blocks coupled. One-third of the resin is removed and the urea cyclization carried out as described above for the first one-third of the library. The last third of the resin is mixed and split once more, 20 building blocks coupled and the urea cyclization carried out.

For variation B, in the first randomized step, the 20 building blocks are coupled to the spacer or PII linker. After resin mixing and Fmoc deprotection, the six amines (8, 39-43) are coupled. After mixing and Fmoc deprotection, 20 building blocks are coupled. Half of the resin is removed and the urea cyclization is carried out as described previously. The Fmoc group on the remaining half of the resin is removed and 20 building block coupled. The urea cyclization is carried out as described previously. After each coupling and deprotection step, the resin is washed with DMF (10×). After completion of synthesis, any other acid labile protecting groups are removed by treatment with 85% TFA containing 2% triisopropylsilane, 2.5% EDT, 5% thioanisole, 5% water for 1-2.5 hours. Then the resin is washed with 90% aqueous acetic acid (4×5 minutes), DMF (2×2 minutes), 5% DIPEA in DMF (2×2 minutes), DMF (4×2 minutes), CH₂Cl₂ (10×2 minutes), and finally methanol (5×2 minutes), before being dried by lyophilization overnight.

### Example 36

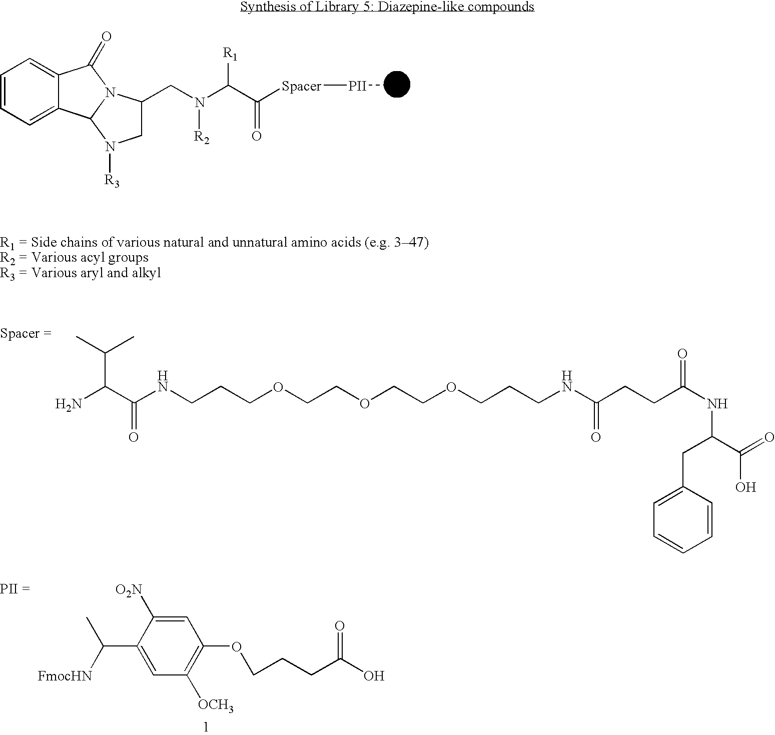

Library 5, shown above, is prepared on PEGA₄₀00 resin (2 g, 0.1 mmol/g; 500-700 μm beads) as shown below in Scheme 13. The library is designed to create diazepine-like templates (when n=3).

Many benzodiazepines have potent biological activities (see Pigeon et al, 1998, Tetrahedron, 54: 1497-1506). As for libraries, 1-4, no single particular protein is targeted and the building blocks used are chosen arbitrarily, but such that as many functional groups as possible were presented in the side chains: e.g. carboxylic acids, amines, indoles, pyridines, aliphatics, aromatics, imidazoles, hydroxyls. For R₁, the building blocks used are judiciously chosen, for example, from compounds 3-47 shown in Tables 1-3 above. Compounds containing Boc-protected amines as a side chain are unsuitable for the first position. R₂ comprises various acyl groups, while R₃ is aryl or alkyl.

The photolabile linker, 1 (3 equivalents) is coupled under TBTU activation. As discussed above for Example 34, Library 5 can be synthesized by the ladder method or without ladder and spacer. In one embodiment of Library 5, a spacer molecule facilitates identification of active ligands by MALDI-MS. In another embodiment of the library, the spacer is not used and the active ligands can be identified using Magic Angle Spinning (MAS)-NMR and/or Tandem mass spectrometry. When a spacer is used, can be produced by sequential coupling of Fmoc-Phe-OH, compound 2, and Fmoc/Boc-Val-OH after TBTU preactivation. When no spacer is used, the first set of randomized amino acids is coupled directly to the photolabile linker. Randomized positions of the library are generated using the split and mix approach described in Furka et al., 1991, Int. J. Peptide Protein Res., 37: 487-493 and Lam et al., 1991, Nature, 354: 82-84, in one or more 20-well custom-made (2.0 mL capacity) multiple column library generators.

In the synthesis of Library 5, the first building block (3 equivalents) is coupled to the spacer or photolabile linker using TBTU/NEM activation. After mixing and cleavage of the Fmoc protecting group by treatment with 20% piperidine in DMF for 4+16 minutes, the amino group is reductively alkylated using Fmoc protected amino aldehydes (49-52) shown in Scheme 13. The synthesis of the amino aldehydes and the solid phase reductive alkylation is carried out as described in St. Hilaire et al, 2002, J. Med. Chem. 45: 1971-1982. To accomplish the reductive alkylation, the resin is first washed with a solution of TEOF containing 1% HOAc (6×). Then, aldehydes (7 equivalents) dissolved in (DMF/TEOF/MeOH (1:1:1)) containing 1% HOAc, is added. After 45 minutes at 50° C., NaCNBH₃ (10.5 equivalents) in (DMF/TEOF/MeOH (1:1:1)) containing 1% HOAc is added and the mixture reacted for an additional 2.5 hours. After completion of coupling, the resin is washed with DMF (2×) MeOH (2×) and DMF (2×).

After mixing, the resulting secondary amines are then acylated using a variety of commercially available acid and sulfonyl chlorides (R₂). After mixing, the Boc protecting group of the amino side chain is then removed by treatment with 20% TFA in CH₂Cl₂ for 20 minutes. The resin is then washed with TEOF containing 1% HOAc (2×) and then reductively alkylated using a variety of commercially available aldehydes to give R₃. The resulting secondary amine is protected by treatment with 20% (Boc)₂O in DMF for 1 hour. After cleavage of the Fmoc group, the N-terminal amine is reacted with TBTU activated compound 53 (4 equivalents) in DMF at room temperature for 1 hour. After washing with DMF (6×), the resin is heated to at 110° C. in DMF for 2 hours to effect cleavage of the Boc protecting group and concomitant cyclization to form the diazepines.

### Example 37

### Results for Myocytes/Ligand Libraries 4 and 5

Using the procedures described above for Examples 1-5, the ligand libraries 4 and 5 as described for Examples 35 and 36, myocyte proteins prepared and labeled as described for Examples 9 and 11, screening as described for Example 15, sorting and identifying as described for Examples 18 to 21, digestion and protein identification as described for Examples 25-31, previously unknown, specific, differential ligand-protein binding pairs are identified for the normal (basal) myocyte protein mixtures and the phenylephrine (PE)-treated myocyte proteins screened against the ligands of Libraries 4 and 5. It is expected that for Library 4, many kinases and their ligand binding partner(s) will be identified. Kinases are, however, not the only class of protein to be identified. With Library 5, no particular class of protein binding is expected.

This disclosure includes numerous literature and patent citations, each which is hereby incorporated by reference for all purposes. The invention is meant to be broadly construed and defined in the following claims.

We claim:

1. A process for identifying specific members of a previously unknown protein-ligand binding pair, comprising the steps of:
(a) synthesizing a ligand library onto resin beads to form an immobilized ligand library, wherein each bead of the immobilized library comprises one member of the ligand library;
(b) incubating the immobilized ligand library with one or more protein mixture;
(c) detecting an immobilized ligand-protein binding pair from the incubation mixture;
(c) identifying the ligand of the ligand-binding pair; and
(d) identifying the protein of the ligand-binding pair;
wherein the identified ligand and protein are specific members of a previously unknown ligand-protein binding pair.

2. The process of claim 1, wherein specific members of a differential ligand-protein binding pair are identified, the process comprising:
(a) incubating the immobilized ligand library with two or more differentially labeled protein mixtures;
(b) detecting a ligand of the library that binds differentially with a protein of the labeled protein mixtures to form a differential ligand-protein binding pair;
(c) identifying the ligand of the specific ligand-binding pair; and
(d) identifying the protein of the differential ligand-protein binding pair, wherein the identified ligand and protein are specific members of a previously unknown differential ligand-protein binding pair.

3. The process of claim 1, wherein said resin is hydrophilic and comprises polyethylene glycol.

4. The process of claim 3, wherein the resin is PolyEthyleneGlycol Acrylamide copolymer (PEGA), Super Permeable Organic Combinatorial Chemistry (SPOCC) or PolyOxyEthylene-PolyOxyPropylene (POEPOP) resin.

5. The process of claim 3, wherein the ligand library comprises a parallel array of random modifications of one or more ligand.

6. The process of claim 5, wherein said library comprises a parallel array of random modifications of a known compound and wherein said protein mixture comprises protein not previously known to bind said compound.

7. The process of claim 1, wherein each protein mixture is not labeled prior to incubation with the ligand library, and wherein each ligand-protein binding pair is detected after incubation by addition of a detection probe.

8. The process of claim 7, wherein the detection probe is silver.

9. The process of claim 1, wherein each protein mixture is labeled with a detection probe, and wherein each ligand-protein binding pair is detected by detection of the probe.

10. The process of claim 9, wherein the detection probe produces fluorescence.

11. The process of claim 10, wherein the detection probe is Oregon Green 514, anthranilic acid, Rhodamine red, or Green Fluorescent Protein (GFP).

12. The process of claim 7, wherein the detection probe produces chemoluminescensce.

13. The process of claim 12, wherein the detection probe is luciferase or aequorin.

14. The process of claim 7, wherein the detection probe produces radioactivity.

15. The process of claim 7, wherein the detection probe is an affinity probe.

16. The process of claim 15, wherein the detection probe is biotin.

17. The process of claim 1, wherein the mixture of proteins is a mixture of mammalian tissue cell proteins.

18. The process of claim 1, wherein the protein mixture is a mixture of viral proteins.

19. The process of claim 1, wherein the protein mixture is a mixture of bacterial proteins.

20. The process of claim 1, wherein the protein mixture is a mixture of fungal proteins.

21. The process of claim 1, wherein the protein mixture is a mixture of protozoan proteins.

22. The process of claim 1, wherein the protein mixture is a mixture of mammalian proteins.

23. The process of claim 1, wherein the protein mixture is a mixture of human proteins.

24. The process of claim 1, wherein the protein mixture is a mixture of plant proteins.

25. The process of claim 1, wherein the protein mixture comprises proteins expressed in a cellular system from a cDNA library that is tagged with a genetic label.

26. The process of claim 25, wherein the genetic label is myc or a photoprotein.

27. The process of claim 1, wherein the ligand library is a peptide library.

28. The process of claim 27, wherein the ligand library comprises glycopeptides.

29. The process of claim 27, wherein the ligand library comprises lipopeptides.

30. The process of claim 27, wherein the ligand library comprises modified peptide scaffolds.

31. The process of claim 1, wherein the ligand library comprises peptidomimetics.

32. The process of claim 1, wherein the ligand library comprises small organic molecules.

33. The process of claim 1, wherein the ligand library comprises oligosaccharides.

34. The process of claim 1, wherein the ligand comprises DNA molecules.

35. The process of claim 1, wherein the ligand library comprises RNA molecules.

36. The process of claim 1, wherein the protein mixture comprises a family of proteins, and wherein the ligand-protein binding pair is detected by immunoassay.

37. The process of claim 1, wherein the ligand is identified by mass spectrometry.

38. The process of claim 37, wherein the ligand is identified by NMR spectroscopy.

39. The process of claim 1, wherein the protein is identified by mass spectrometry.

40. The process of claim 1, further comprising isolating a resin bead containing the immobilized ligand-protein binding pair from the incubation mixture, wherein the steps of identifying the ligand and identifying the protein are carried out on the isolated resin bead.

41. An isolated ligand-protein binding pair comprising:
a) Pip-Pal-Pal-Phe-Pya-Pip [SEQ ID NO: 7]; and
b) Myosin light chain kinase, Tyrosine phosphatase, ATP Synthase component, Gluthathione S-transferase, Cytochrome P450, (60s) Ribosomal protein, or SPTR.

42. An isolated ligand-protein binding pair comprising:
a) Pya-Hyp-Hyp-Phe-Acm-Tyr [SEQ ID NO: 8]; and
b) Troponin T, Growth hormone receptor, or Protein kinase.

43. An isolated ligand-protein binding pair comprising:
a) Pya-Gua-Pip-Acc-Phe-Pip [SEQ ID NO: 9]; and
b) NADH dehydrogenase, ATP binding component, Myosin, or Histone associated protein.

44. An isolated ligand-protein binding pair comprising:
a) Phe-Aze-Gly-His-Gly-Aze [SEQ ID NO: 10]; and
b) Mitochondrial ATP synthase, Ribosomal protein (L series), Serine protease, or SPTR.

45. An isolated ligand-protein binding pair comprising:
a) Phe-Thr-Pya-Pip-Asp-His [SEQ ID NO: 11]; and
b) Sodium channel, Chloride channel, Troponin, Ribosomal protein L26, Serine hydroxy methyl transferase, Zinc Finger protein, Adherin aminotransferase, Gluthathione transferase, or Gluthathione peroxidase.

46. An isolated ligand-protein binding pair comprising:
a) Phe-Ppy-Acc-Ala-Ppy-Hpy [SEQ ID NO: 12]; and
b) Troponin T, Phospholipase C, or Phosphatidylcholine sterol acyl transferase.

47. An isolated ligand-protein binding pair comprising:
a) Phe-Abi-Pal-Hyp-Thr-Hyp [SEQ ID NO: 13]; and
b) Zinc finger associated protein, Ribosomal proteins, or Protein phosphatase.

48. An isolated ligand-protein binding pair comprising:
a) Phe-Gua-Pal-Tyr-Gua-Tyr [SEQ ID NO: 14]; and
b) Glucose-6-Phosphatase, Succinate dehydrogenase, ARL-interacting protein, SPTR, or Nucleic acid binding protein.

49. An isolated ligand-protein binding pair comprising:
a) Pal-Abi-Gly-Gly-Abi-His [SEQ ID NO: 15]; and
b) 60s Ribosomal protein, 40s Ribosomal protein, or Low density lipoprotein receptor.

50. An isolated ligand-protein binding pair comprising:
a) Abi-Thr-Hyp-Hyp-His-?-[SEQ ID NO: 16]; and
b) Phosphofructokinase, Selenium binding protein, Serine arginine rich protein kinase, Guanylate kinase, Protein tyrosine kinase, Alkaline phosphatase, Symporter, SPTR, WAP-protein, GTP Hydrolase, or Actin filament.

51. An isolated ligand-protein binding pair comprising:
a) Pya-Gua-Abi-Asp-Abi-Tyr [SEQ ID NO: 17]; and
b) SPTR, 60s Ribosomal protein, Calcium channel, Slo channel protein isoform, Potassium conductance calcium activated channel, Symporter, NADH dehydrogenase, Malate dehydrogenase, N-Acetyl transferase, Mitochondrial associated protein, or G-protein signaling receptor.

52. An isolated ligand-protein binding pair comprising:
a) Abi-Phe-Abi-Phe-Che-Tyr [SEQ ID NO: 18]; and
b) Cathepsin E, Ribosomal protein, Actin binding protein, or Amino acid transferase.

53. An isolated ligand-protein binding pair comprising:
a) T(Sa)-F—N—H—S [SEQ ID NO: 19]; and
b) Phosphate acetyl transferase, acid shock protein, or molybdopterin converting factor subunit.

54. An isolated ligand-protein binding pair comprising:
a) T(Sa)-F-A-L-V [SEQ ID NO: 20]; and
b) Chaperone DnaK or transposase.

55. An isolated ligand-protein binding pair comprising:
a) T(Sa)-F-G-I—W [SEQ ID NO: 21]; and
b) Histidine synthetase or aspartate carbamoyl transferase.

56. An isolated ligand-protein binding pair comprising:
a) T(Sa)-F-G-I-M [SEQ ID NO: 22]; and
b) Transposase, transcriptional regulator, or GroEL.

57. An isolated ligand-protein binding pair comprising:
a) T(Sa)-G-V-F-L [SEQ ID NO: 23]; and
b) 50 S ribosomal protein, heme binding lipoprotein, regulator for D-glucarate, D-glycerate and D-galactarate, or glutamine tRNA synthetase.

58. An isolated ligand-protein binding pair comprising:
a) T(Sa)-Y—S-M-P [SEQ ID NO: 24; and
b) Biotin synthetase, UDP-glucose dehydrogenase, tyrosine protein kinase, or fatty acid oxidase complex proteins.

59. An isolated ligand-protein binding pair comprising:
a) T(Sa)-L-S—W—W [SEQ ID NO: 25]; and
b) NAD-dependent 7-alpha-hydroxysteriod dehydrogenase, homocysteine transferase, nitrate reductase, lactate dehydrogenase, or citrate synthetase.

60. An isolated ligand-protein binding pair comprising:
a) T(Sa)-H—W—H—I [SEQ ID NO: 26]; and
b) Mannose-1-phosphate guanyl transferase or isopropyl malate dehydrogenase.

61. An isolated ligand-protein binding pair comprising:
a) T(Sa)-H—W—V—V [SEQ ID NO: 27]; and
b) Pyruvoyl dependent aspartate decarboxylase, colicin E2, or Histidine kinase.

62. An isolated ligand-protein binding pair comprising:
a) T(Sa)-H-L-G-Y [SEQ ID NO: 28]; and
b) phosphomannose isomerase.

63. An isolated ligand-protein binding pair comprising:
a) T(Sa)-I—Y-L-F [SEQ ID NO: 29]; and
b) Membrane bound ATP synthetase or ATP hydrolase.

64. An isolated ligand-protein binding pair comprising:
a) T(Sa)-F-G-L-M [SEQ ID NO: 30]; and
b) Hemolysin C, high affinity potassium transport system, quinone oxidoreductase, or ferrodoxin dependent NA(D)PH oxidoreductase.

65. An isolated ligand-protein binding pair comprising:
a) T(Sa)-W—V—N-M [SEQ ID NO: 31]; and b) Transposase or inner membrane protein for phage attachment.

66. An isolated ligand-protein binding pair comprising:
a) T(Sa)-M-V-N—W [SEQ ID NO: 32; and
b) ATP dependent helicase or mob C.

67. An isolated ligand-protein binding pair comprising:
a) T(Sa)-H—I-G-Y [SEQ ID NO: 33]; and
b) Fimbrial subunit or outer membrane pyruvate kinase.

68. An isolated ligand-protein binding pair comprising:
a) T(Sa)-L-Y-L-F [SEQ ID NO: 34]; and
b) Fimbrial protein precursor, alkaline phosphatase, cytochrome related proteins.

69. An isolated ligand-protein binding pair comprising:
a) T(Sa)-H—W—H-L [SEQ ID NO: 35]; and
b) Chorismate mutase, xanthine dehydrogenase, or carbamoyl phosphate synthetase.

70. An isolated ligand-protein binding pair comprising:
a) T(Sa)-F—V—W—H [SEQ ID NO: 36]; and
b) NADH dehydrogenase, protein involved in flagellar biosynthesis and motor switching component, Lysine-arginine-ornithine-binding protein, or ATP-binding component of glycine-betaine-proline transport protein.

71. An isolated ligand-protein binding pair comprising:
a) T(Sa)-L-Y—I—F [SEQ ID NO: 37]; and
b) Colicin, outer membrane lipoprotein, or arylsulfatase.

72. An isolated ligand-protein binding pair comprising:
a) ManS-Gly-ManS-Asp-Asn-Ala [SEQ ID NO: 38]; and
b) Con A or P. sativum lectin.

73. An isolated ligand-protein binding pair comprising:
a) ManS-Gly-GIcNN-Asn-ManS-Tyr [SEQ ID NO: 39]; and
b) Con A, P. sativum lectin, or L. culinaris lectin.

74. An isolated ligand-protein binding pair comprising:
a) ManN-Phe-Trp-Ser-Lys-His [SEQ ID NO: 40]; and
b) Con A or P. sativum lectin.

75. An isolated ligand-protein binding pair comprising:
a) GlcNN-Trp-Phe-Asp-Trp-Pro [SEQ ID NO: 41]; and
b) Con A.

76. An isolated ligand-protein binding pair comprising:
a) GlcNN-Val-GIcNN-His-ManS-Gly [SEQ ID NO: 42]; and
b) Con A or P. sativum lectin.

77. An isolated ligand-protein binding pair comprising:
a) ManN-ManS-ManN-Trp-Ser-Trp [SEQ ID NO: 43]; and
b) Con A, P. sativum lectin, or L. culinaris lectin.

78. An isolated ligand-protein binding pair comprising:
a) Gly-Pro-Lys-Lys-Tyr-His [SEQ ID NO: 44]; and
b) Con A, P. sativum lectin, or L. culinaris lectin.

79. An isolated ligand-protein binding pair comprising:
a) His-Thr-Trp-Gly-Tyr-Trp [SEQ ID NO: 45]; and
b) Con A.
